(19) United States
(12) Patent Application Publication
(10) Pub. No.: US 2025/0226083 A1
(43) Pub. Date: Jul. 10, 2025

(54) MEDICAL INSPECTION DEVICE MANAGEMENT SYSTEM, ULTRASONIC DIAGNOSIS APPARATUS AND MANAGEMENT APPARATUS

(71) Applicant: CANON MEDICAL SYSTEMS CORPORATION, Otawara-shi (JP)

(72) Inventors: Masao TAKIMOTO, Otawara (JP); Shigemitsu NAKAYA, Nasushiobara (JP); Yoshinori GOTO, Nasushiobara (JP); Takeshi FUKASAWA, Nasushiobara (JP); Koichi MATSUSHITA, Nasushiobara (JP)

(73) Assignee: CANON MEDICAL SYSTEMS CORPORATION, Otawara-shi (JP)

(21) Appl. No.: 18/999,959

(22) Filed: Dec. 23, 2024

(30) Foreign Application Priority Data
Jan. 9, 2024 (JP) 2024-001477

(51) Int. Cl. G16H 40/20; A61B 8/00; G16H 10/60; G16H 40/67

(52) U.S. Cl. CPC G16H 40/20; A61B 8/4405; G16H 10/60; G16H 40/67; A61B 8/461; A61B 8/467

(57) ABSTRACT

A medical inspection device management system according to the present embodiment comprises one or a plurality of medical inspection devices having a self-traveling function of moving to a destination and a management apparatus, wherein the management apparatus includes a first processing circuitry configured to select an available medical inspection device based on an inspection order, set the destination of the selected medical inspection device, and transmit destination information regarding the destination to the selected medical inspection device.

### CROSS REFERENCE TO RELATED APPLICATIONS

This application is based upon and claims the benefit of priority from the prior Japanese Patent Application No. 2024-001477, filed on Jan. 9, 2024, the entire contents of which are incorporated herein by reference.

### FIELD

Embodiments described in the present specification and drawings relate to a medical inspection device management system, an ultrasonic diagnosis apparatus and a management apparatus.

### BACKGROUND

In related art, in a hospital such as an advanced treatment hospital, there are a case where an ultrasonic diagnosis apparatus is integrated in a predetermined inspection room and is operated in common by diagnosis and treatment departments in the hospital and a case where ultrasonic diagnosis apparatuses are independently purchased in diagnosis and treatment departments in the hospital and are operated in the diagnosis and treatment departments in the hospital. Thus, although there are a large number of ultrasonic diagnosis apparatuses in the hospital, since an administrator and an installation place are different between the ultrasonic diagnosis apparatus operated in common by the diagnosis and treatment departments in the hospital and the ultrasonic diagnosis apparatuses independently purchased in the diagnosis and treatment departments in the hospital, it is difficult to grasp a total number of ultrasonic diagnosis apparatuses. In addition, in a case where the ultrasonic diagnosis apparatus independently purchased in the diagnosis and treatment department is used in another diagnosis and treatment department, a staff member needs to move the ultrasonic diagnosis apparatus from the diagnosis and treatment department to a predetermined inspection room.

In recent years, along with a low birth rate and an aging society, a problem of a staff member shortage has occurred in hospitals. Thus, in hospital management and hospital operation, there is a demand for effective utilization of assets and resources and further improvement in productivity of medical workers. However, since it is difficult to use an ultrasonic diagnosis apparatus of another diagnosis and treatment department having different management or to find and use an ultrasonic diagnosis apparatus having a poor operating rate, it is difficult to effectively use a large number of ultrasonic diagnosis apparatuses present in the hospital. In addition, even though it is possible to rent an ultrasonic diagnosis apparatus from another diagnosis and treatment department, since the movement of the ultrasonic diagnosis apparatus requires the labor of the staff member, it is not efficient in a situation where the problem of the staff member shortage occurs.

In addition, these problems occur not only in the ultrasonic diagnosis apparatus but also in other medical inspection devices such as a mobile X-ray diagnosis apparatus, a mobile magnetic resonance imaging (MRI) apparatus, and an endoscope apparatus. Therefore, it is desired to improve the productivity of the medical workers while effectively utilizing the assets and resources in the hospital by collectively the managing medical inspection devices present in the hospital.

### BRIEF DESCRIPTION OF THE DRAWINGS

FIG. 1 is a diagram for describing an outline of a medical inspection device management system according to a first embodiment;

FIG. 2 is a diagram illustrating an example of a destination of a medical inspection device in the medical inspection device management system according to the first embodiment;

FIG. 3 is a cycle diagram for describing a series of flows when the medical inspection device is used for an inspection in the medical inspection device management system according to the first embodiment;

FIG. 4 is a block diagram illustrating a configuration example of the medical inspection device management system according to the first embodiment;

FIG. 5 is a schematic view illustrating an appearance of the medical inspection device according to the first embodiment;

FIG. 6 is a block diagram illustrating an example of an electrical configuration of the medical inspection device according to the first embodiment;

FIGS. 7A and 7B are diagrams illustrating an example of a non-contact power supply provided in a hospital in the first embodiment;

FIG. 8 is a diagram illustrating an example of transmission and reception of medical data and setting data in the first embodiment;

FIGS. 9A and 9B are schematic views illustrating an appearance at the time of use and at the time of movement of the medical inspection device according to the first embodiment;

FIG. 10 is a block diagram illustrating an example of a configuration of a management apparatus according to the first embodiment;

FIG. 11 is a diagram illustrating an example of an inspection reservation table stored in a memory of the management apparatus according to the first embodiment;

FIG. 12 is a diagram illustrating an example of a floor map stored in a memory of the management apparatus according to the first embodiment;

FIG. 13 is a flowchart for describing contents of reservation processing executed by the management apparatus according to the first embodiment;

FIG. 14 is a diagram illustrating an example of an inspection order received by the management apparatus according to the first embodiment;

FIG. 15 is a flowchart for describing an example of movement instruction processing executed in the management apparatus according to the first embodiment;

FIG. 16 is a flowchart for describing an example of the movement instruction processing executed in the management apparatus according to the first embodiment;

FIG. 17 is a diagram illustrating an example of a floor map on which a position of the medical inspection device is displayed in the first embodiment;

FIG. 18 is a diagram illustrating the medical inspection device moved to a sterilization room in the first embodiment;

FIG. 19 is a diagram illustrating the medical inspection device moved to a fixture room in the first embodiment;

FIG. 20 is a flowchart for describing contents of movement control processing executed by the medical inspection device according to the first embodiment;

FIG. 21 is a flowchart for describing contents of data transmission processing executed by the medical inspection device according to the first embodiment;

FIG. 22 is a flowchart for describing contents of diagnosis processing executed by the medical inspection device according to the first embodiment;

FIG. 23 is a diagram illustrating an example of a reservation status list displayed on a display of a terminal apparatus of a medical inspection device management system according to Modification 1;

FIG. 24 is a diagram illustrating another example of an inspection order received by a management apparatus according to Modification 2; and

FIGS. 25A and 25B are schematic views illustrating another example of an appearance at the time of use and at the time of movement of the medical inspection device according to the first embodiment.

### DETAILED DESCRIPTION

Hereinafter, respective embodiments of the medical inspection device management system, the ultrasonic diagnosis apparatus and the management apparatus will be described with reference to the accompanying drawings. In the embodiments below, the same reference signs are given for identical components in terms of configuration and function, and duplicate description is omitted.

### Outline of First Embodiment

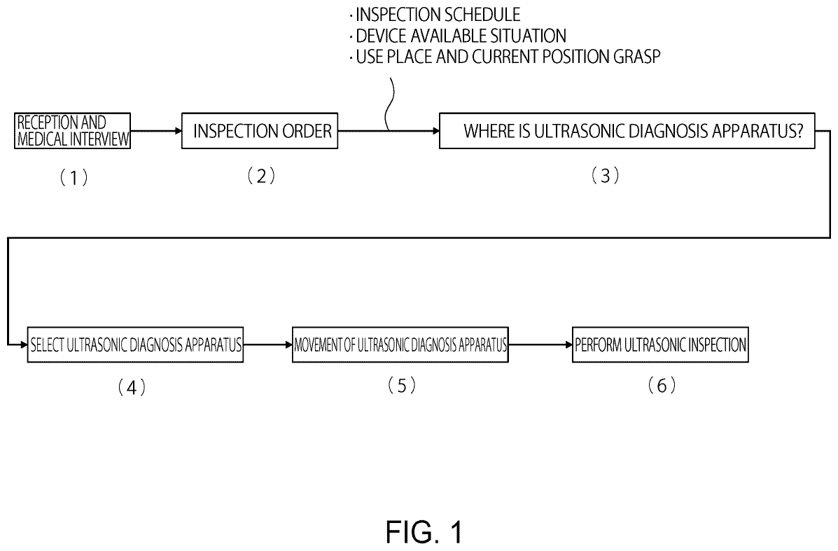

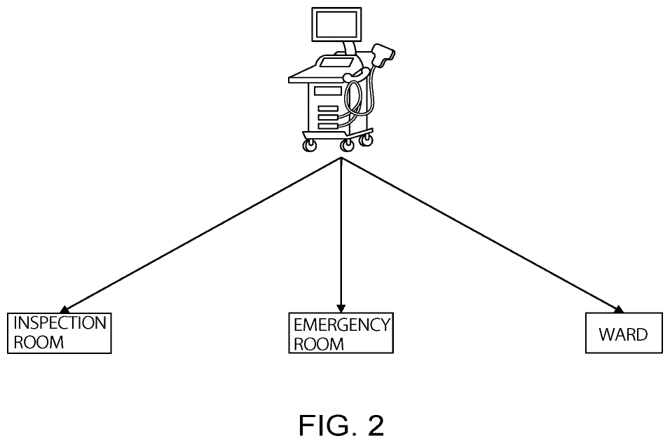

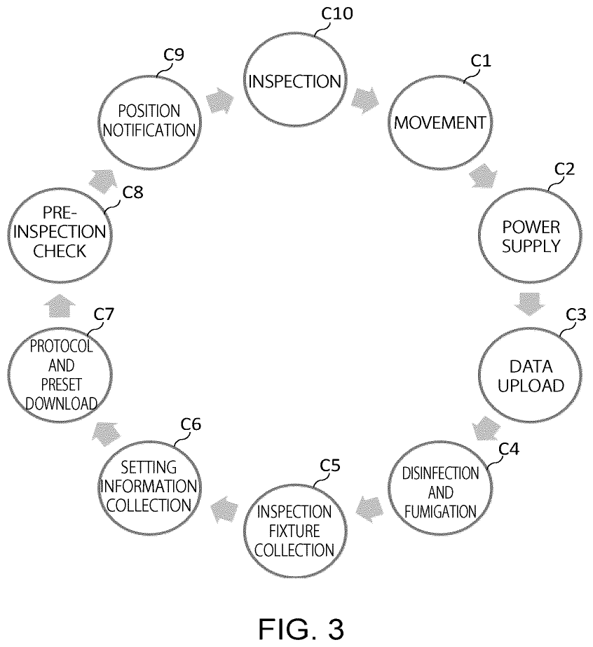

First, an outline of a medical inspection device management system according to a first embodiment will be described with reference to FIGS. 1 to 3. FIG. 1 is a diagram for describing an outline of the medical inspection device management system according to the first embodiment. FIG. 2 is a diagram illustrating an example of a destination of a medical inspection device in the medical inspection device management system according to the first embodiment. FIG. 3 is a cycle diagram for describing a series of flows when the medical inspection device is used for an inspection in the medical inspection device management system according to the first embodiment. As illustrated in FIG. 1, in the present embodiment, a case where the medical inspection device is an ultrasonic diagnosis apparatus will be described as an example. Note that, hereinafter, a doctor and an inspection technician are collectively referred to as a user, and a doctor, an inspection technician, a nurse, a staff member, and the like are collectively referred to as a medical worker as necessary.

As illustrated in FIG. 1, in (1) and (2), first, the user such as the doctor or the inspection technician performs a medical interview (or medical inspection) on a patient. Then, in a case where the user determines that it is necessary to perform ultrasonic inspection during the medical interview or the medical inspection, the user orders inspection. Note that, the inspection is an inspection performed on a subject, and means the ultrasonic inspection in the present embodiment. In (3) and (4), a management apparatus selects an available ultrasonic diagnosis apparatus from, for example, an inspection schedule, availability of a device, a use place, a current position of the ultrasonic diagnosis apparatus, and the like based on the inspection order. As illustrated in FIG. 2, the use place of the ultrasonic diagnosis apparatus includes, for example, an inspection room where various inspections such as ultrasonic inspection are performed, an emergency room where a patient accepted in an emergency visit is present, a ward (hospital room) where a hospitalized patient is present, and the like. In (5) and (6), when an inspection date and time based on the inspection order comes, the management apparatus transmits destination information regarding the destination to the selected ultrasonic diagnosis apparatus. The ultrasonic diagnosis apparatus having received the destination information moves by self-traveling (automatic traveling) in a hospital to the destination based on the destination information, and notifies the medical worker that the ultrasonic diagnosis apparatus arrives at the destination. Then, the user performs the ultrasonic inspection on the patient at the destination by using the ultrasonic diagnosis apparatus.

That is, as illustrated in FIG. 3, after starting moving (C1), the ultrasonic diagnosis apparatus charges a battery with power supplied from a non-contact power supply provided in the hospital (C2), and uploads medical data collected in a previous inspection, preset data used in the previous inspection, and protocol data to a data server (C3) at the same time. Then, the ultrasonic diagnosis apparatus is disinfected and fumigated (C4), and medical inspection fixtures necessary for a next inspection are collected (C5). Subsequently, the ultrasonic diagnosis apparatus downloads the protocol data and the preset data as collection of setting data while moving to the destination (C6 and C7). In addition, while moving to the destination, the ultrasonic diagnosis apparatus performs pre-inspection check of an ultrasonic diagnosis apparatus body and a medical inspection fixture such as an ultrasonic probe electrically connected to the ultrasonic diagnosis apparatus body (C8). Then, in a case where the ultrasonic diagnosis apparatus arrives at the destination, the management apparatus notifies the medical worker that the ultrasonic diagnosis apparatus arrives (C9). As described above, the user performs the ultrasonic inspection on the patient at the destination by using the arrived ultrasonic diagnosis apparatus (C10). In addition, after the end of the inspection, when there is a next inspection, the ultrasonic diagnosis apparatus repeats a cycle of C1 to C10. As a result, since the management apparatus can cause the available ultrasonic diagnosis apparatus in the hospital to arrive at the inspection room without the medical worker moving the ultrasonic diagnosis apparatus, the productivity of the medical worker can be improved while effectively utilizing the assets in the hospital.

### Detailed Description according to First Embodiment

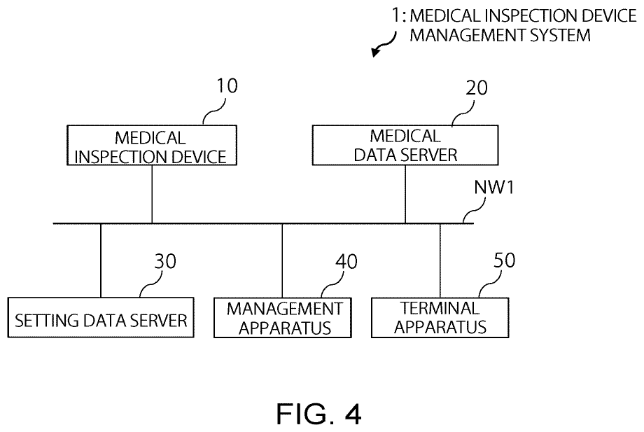

Next, a configuration example of the medical inspection device management system according to the present embodiment will be described in detail with reference to FIG. 4. FIG. 4 is a block diagram illustrating a configuration example of the medical inspection device management system according to the first embodiment. As illustrated in FIG. 4, a medical inspection device management system 1 according to the present embodiment includes a medical inspection device 10, a medical data server 20, a setting data server 30, a management apparatus 40, and a terminal apparatus 50. The medical inspection device 10, the medical data server 20, the setting data server 30, the management apparatus 40, and the terminal apparatus 50 are connected to communicate via an in-hospital network NW1 by a dedicated line in the hospital. Note that, the medical inspection device 10, the medical data server 20, the setting data server 30, the management apparatus 40, and the terminal apparatus 50 may be connected to communicate with each other via a network via a public line such as the Internet.

The medical inspection device 10 is a device for medically inspecting the patient. The medical inspection device 10 is, for example, an ultrasonic diagnosis apparatus used for an ultrasonic inspection or a Fibroscan inspection, an X-ray computed tomography (CT) apparatus used for a CT inspection, a magnetic resonance imaging (MRI) apparatus used for a MRI inspection, a mammography apparatus used for a mammography inspection, an endoscope inspection apparatus used for an endoscope inspection, a nuclear medicine diagnosis apparatus, or the like. The medical inspection device 10 has a self-traveling function of moving to the destination. That is, the medical inspection device 10 moves by automatically traveling to the destination such as the inspection room, the emergency room, or the hospital room in the hospital, for example. Note that, in the following description, the medical inspection device management system 1 will be described by using a case where the medical inspection device 10 is used in the inspection room, the emergency room, the hospital room, or the like in the hospital as an example, but a place where the medical inspection device 10 is used is not limited thereto. That is, the place where the medical inspection device 10 is used is arbitrary, and for example, the medical inspection device may be used in a medical helicopter, a patrol medical inspection, or a mobile medical care vehicle used in regional medical care. As described above, in a case where The medical inspection device 10 is used in the medical helicopter or the mobile medical care vehicle, a place where the medical helicopter is stopped, a parking position of the mobile medical care vehicle, and the like are set as the destination. In addition, as described above, in the following description, a case where the medical inspection device 10 is the ultrasonic diagnosis apparatus will be described as an example.

The medical data server 20 is a server that saves the medical data. Specifically, the medical data server 20 receives the medical data from the medical inspection device 10 via the in-hospital network NW1, and stores and saves the medical data in a storage circuitry in the medical data server. For example, the medical data server 20 is realized by a picture archiving and communication system (PACS) or the like, and saves a medical image in a format conforming to digital imaging and communications in medicine (DICOM). Here, the medical data includes medical image data collected by the medical inspection device 10 in the inspection and data such as various measurement results and diagnosis reports performed on the medical image data.

The setting data server 30 is a server that saves the setting data. Specifically, the setting data server receives the setting data from the medical inspection device 10 via the in-hospital network NW1, and saves the setting data for each user in a storage circuitry in the setting data server. Here, the setting data is data set for each user, and includes the protocol data and the preset data. The protocol data is data regarding a procedure of the inspection performed by the user by using the medical inspection device 10 in order for the user to diagnose the subject, and is data set in advance such that the user themselves can easily perform the inspection. In addition, the preset data is data regarding image quality setting when medical image data is displayed, data such as key arrangement of an operation panel in an input interface to be described later, and the like.

The management apparatus 40 is an apparatus that manages the medical inspection device 10. Specifically, the management apparatus 40 manages the medical inspection device 10 by acquiring positional information from one or a plurality of medical inspection devices 10 present in the hospital or transmitting destination information to one or a plurality of medical inspection devices 10 present in the hospital. Note that, it is assumed that the management apparatus 40 is, for example, a personal computer (PC) or a computer such as a server apparatus. In addition, the management apparatus 40 may be provided in a cloud environment, or may be installed in a facility other than a medical institution such as a hospital or a clinic.

The terminal apparatus 50 is a terminal operated by the user such as the doctor or the inspection technician. The terminal apparatus 50 accepts the inspection order from the user and transmits the accepted inspection order to the management apparatus 40 or the like via the in-hospital network NW1.

Here, the inspection order is information input by the user at the time of inspection reservation. The inspection order includes, for example, a user name corresponding to a name of a user, medical inspection device information regarding a medical inspection device used in the inspection, an inspection content, an inspection place such as an inspection room name, an inspection date and time, patient information such as a patient ID and a patient name, information for identifying whether the patient is an outpatient or an inpatient, and inspection number information regarding the number of times of inspections such as a first inspection and return inspections. Note that, the inspection order does not necessarily include all the information described above. That is, the information included in the inspection order is any information. For example, the inspection order transmitted to the management apparatus 40 may include at least the medical inspection device information and the inspection date and time. In addition, the inspection order may include information other than the above-described information. For example, the inspection order may include desired inspection technician information regarding a name of an inspection technician desired by the doctor to perform the inspection, desired medical inspection device information regarding the medical inspection device desired by the user to use, and used fixture information regarding the medical inspection fixture used in the inspection by the user. In addition, the medical inspection fixture is a fixture used in the inspection. For example, in the case of the ultrasonic diagnosis apparatus, the medical inspection fixture is an ultrasonic probe, an ultrasonic jelly, or the like.

In addition, in a case where the medical inspection device management system 1 cooperates with a hospital information system (HIS) or a radiology information system (RIS), the user does not need to input all of the user name, the medical inspection device information, the inspection content, the inspection place, the inspection date and time, the patient information, the information for identifying whether the patient is the outpatient or the inpatient, and the inspection number information. That is, the information input as the inspection order by the user is arbitrary. For example, in a case where the hospital information system has the patient information, the information for identifying whether the patient is the outpatient or the inpatient, and the inspection number information, the user does not need to input the patient information, the information for identifying whether the patient is the outpatient or the inpatient, and the inspection number information.

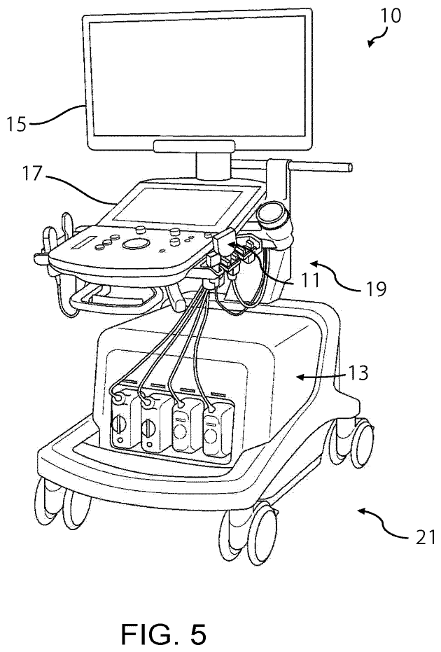

FIG. 5 is a schematic view illustrating an appearance of the medical inspection device 10 according to the first embodiment. As illustrated in FIG. 5, the medical inspection device 10 includes an ultrasonic probe 11, an apparatus body 13, a display 15, an input interface 17, a support member 19, and a movable unit 21.

The ultrasonic probe 11 is a device that is connected to the apparatus body 13, transmits an ultrasonic wave to the subject, and receives a reflected wave signal reflected in the subject based on the transmitted ultrasonic wave. The ultrasonic probe 11 is, for example, a 1D array probe that scans a two-dimensional region in the subject, a mechanical 4D probe or a 2D array probe that scans a three-dimensional region in the subject, or the like. In addition, the ultrasonic probe 11 is connected to the apparatus body 13 by wired communication or wireless communication.

The apparatus body 13 is an apparatus that generates an ultrasonic image based on a reception signal received from the ultrasonic probe 11. In the example illustrated in FIG. 5, the ultrasonic probe 11 is connected to the apparatus body 13 according to the present embodiment. In addition, the display 15 and the input interface 17 are connected to the apparatus body 13 according to the present embodiment via the support member 19. The apparatus body 13 corresponds to a medical inspection device body according to the present embodiment.

The display 15 is a display apparatus that displays various ultrasonic images, various settings, and the like. For example, the display 15 displays an ultrasonic image generated by the apparatus body 13, a graphical user interface (GUI) for accepting various operations from an operator, and the like. In the present embodiment, the display 15 includes, for example, a liquid crystal display, a cathode ray tube (CRT) display, or the like. The display 15 corresponds to a display according to the present embodiment.

The input interface 17 is an input apparatus for performing various settings and the like, and accepts an input operation for operating the apparatus body 13. The input interface 17 is realized by, for example, a trackball, a switch button, a mouse, a keyboard, a touch pad that performs an input operation by touching an operation surface, a touch monitor in which a display screen and the touch pad are integrated, a non-contact input circuitry using an optical sensor, a sound input circuitry, and the like. The input interface 17 is connected to a processing circuitry of the apparatus body 13 to be described later, converts an input operation accepted from the medical worker into an electric signal, and outputs the electric signal to the processing circuitry of the apparatus body 13. Note that, in the present specification, the input interface 17 is not limited to the interface including physical operation components such as a mouse and a keyboard. For example, an electric signal processing circuitry that accepts an electric signal corresponding to an input operation from an external input device provided separately from the apparatus and outputs the electric signal to the processing circuitry of the apparatus body 13 is also included in the example of the input interface 17.

The support member 19 movably supports the display 15 and the input interface 17 with respect to the apparatus body 13. The support member 19 couples the apparatus body 13 to the display 15 and the input interface 17.

The movable unit 21 is provided at a lower part of the apparatus body 13. For example, the movable unit 21 includes wheels and casters. The movable unit 21 is rotated by a drive unit to be described later under the control of a control function in the processing circuitry, and moves the apparatus body 13 and the like on a floor surface.

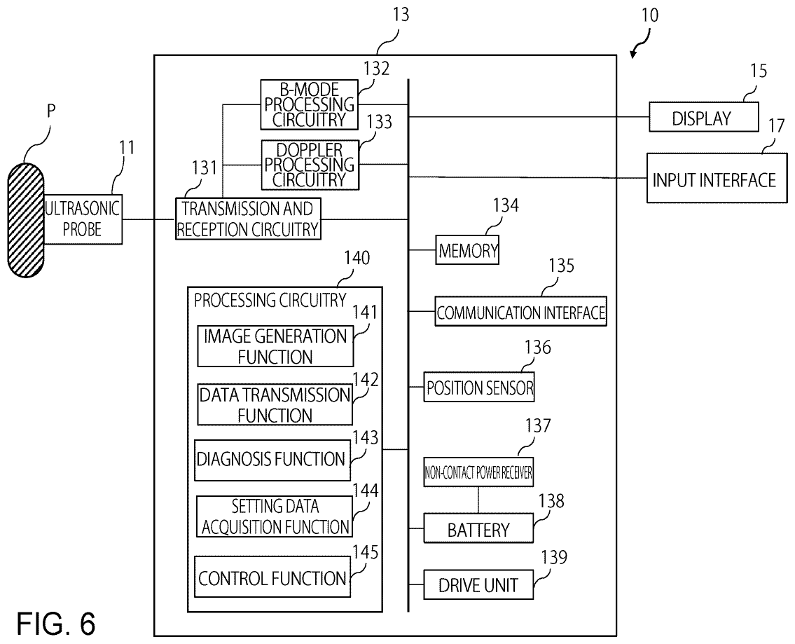

FIG. 6 is a block diagram illustrating an example of an electrical configuration of the medical inspection device 10 according to the first embodiment. As illustrated in FIG. 6, the medical inspection device 10 includes the ultrasonic probe 11, the apparatus body 13, the display 15, and the input interface 17. In addition, the apparatus body 13 according to the present embodiment includes a transmission and reception circuitry 131, a B-mode processing circuitry 132, a Doppler processing circuitry 133, a memory 134, a communication interface 135, a position sensor 136, a non-contact power receiver 137, a battery 138, a drive unit 139, and a processing circuitry 140.

The ultrasonic probe 11 is connected to the transmission and reception circuitry 131. The ultrasonic probe 11 includes, for example, a plurality of piezoelectric vibrators in a probe body, and the plurality of piezoelectric vibrators generate ultrasonic waves based on a drive signal supplied from the transmission and reception circuitry 131. In addition, the ultrasonic probe 11 receives a reflected wave from a subject P and converts the reflected wave into an electric signal. In addition, the ultrasonic probe 11 includes, in the probe body, a matching layer provided on the piezoelectric vibrator, a backing material that prevents propagation of the ultrasonic wave rearward from the piezoelectric vibrator, and the like.

When the ultrasonic wave is transmitted from the ultrasonic probe 11 to the subject P, the transmitted ultrasonic wave is reflected one after another on a discontinuous surface of an acoustic impedance in a body tissue of the subject P, and is received as the reflected wave signal by the plurality of piezoelectric vibrators included in the ultrasonic probe 11. An amplitude of the received reflected wave signal depends on a difference in acoustic impedance at the discontinuous surface from which the ultrasonic wave is reflected. Note that, a reflected wave signal in a case where a transmitted ultrasonic pulse is reflected by a moving blood flow or a surface of a heart wall or the like receives frequency shift depending on a velocity component with respect to an ultrasonic transmission direction of a moving body due to a Doppler effect.

Note that, the present embodiment can be applied to a case where the subject P is scanned two-dimensionally by the ultrasonic probe 11 that is a one-dimensional ultrasonic probe in which the plurality of piezoelectric vibrators are disposed in a line and a case where the subject P is scanned three-dimensionally by the ultrasonic probe 11 that mechanically swings the plurality of piezoelectric vibrators of the one-dimensional ultrasonic probe or the ultrasonic probe 11 that is a two-dimensional ultrasonic probe in which the plurality of piezoelectric vibrators are disposed two-dimensionally in a lattice shape.

The transmission and reception circuitry 131 is a processor that controls transmission directivity and reception directivity in ultrasonic wave transmission and reception, and includes a transmission circuitry and a reception circuitry. Note that, although FIG. 6 illustrates an example of a case where the transmission and reception circuitry 131 is provided in the apparatus body 13, the transmission and reception circuitry 131 may be provided in the ultrasonic probe 11 or may be provided in both the ultrasonic probe 11 and the apparatus body 13.

The transmission circuitry includes a pulse generator, a transmission delay circuitry, a pulser circuitry, and the like, and supplies a drive signal to an ultrasonic vibrator. The pulse generator repeatedly generates rate pulses for forming transmitted ultrasonic waves at a predetermined rate frequency. The transmission delay circuitry gives, to each rate pulse generated by the pulse generator, a delay time for each ultrasonic vibrator necessary for focusing the ultrasonic wave generated from the piezoelectric vibrator in a beam shape and determining transmission directivity. In addition, the pulser circuitry applies a drive pulse to the ultrasonic vibrator at a timing based on the rate pulse. The transmission delay circuitry arbitrarily adjusts a transmission direction of the ultrasonic wave transmitted from an ultrasonic vibrator surface by changing the delay time given to each rate pulse.

The reception circuitry includes an amplifier circuitry, an A/D converter, an adder, and the like, receives a reflected wave signal received by the ultrasonic vibrator, and performs various kinds of processing on the reflected wave signal to generate a reception signal (echo signal). The amplifier circuitry amplifies the reflected wave signal for each channel and performs gain correction processing. The A/D converter performs A/D conversion on a reflected wave signal whose gain is corrected, and gives digital data a delay time necessary for determining reception directivity. The adder performs addition processing of the reflected wave signal processed by the A/D converter to generate a reception signal. By the addition processing of the adder, a reflection component from a direction corresponding to the reception directivity of the reflected wave signal is emphasized.

The B-mode processing circuitry 132 generates B-mode data based on the reception signal received from the transmission and reception circuitry 131. Specifically, for example, the B-mode processing circuitry 132 performs envelope detection processing, logarithmic compression processing, and the like on the reception signal received from the transmission and reception circuitry 131, and generates B-mode data in which signal intensity is expressed by brightness of luminance.

The Doppler processing circuitry 133 generates Doppler data such as a blood flow velocity, blood flow dispersion, and blood flow power based on the reception signal received from the transmission and reception circuitry 131. The Doppler processing circuitry 133 generates Doppler data obtained by extracting blood flow information based on the Doppler effect of the moving body in a region of interest (ROI) set in a scan region by performing frequency analysis on the reception signal received from the transmission and reception circuitry 131.

The memory 134 stores display image data generated by the processing circuitry 140. In addition, the memory 134 can also store data generated by the B-mode processing circuitry 132 or the Doppler processing circuitry 133. In addition, the memory 134 stores control programs for performing ultrasonic wave transmission and reception, image processing, and display processing, diagnosis information (for example, patient ID, doctor's finding, and the like), and various kinds of data such as diagnosis protocols and various body marks.

The communication interface 135 is connected to, for example, the medical data server 20, the setting data server 30, and the management apparatus 40 via the in-hospital network NW1, and performs data communication with the medical data server 20, the setting data server 30, and the management apparatus 40.

The position sensor 136 is a sensor that detects positional information of the medical inspection device 10. The position sensor 136 transmits the detected positional information to the management apparatus 40 via the communication interface 135.

The non-contact power receiver 137 receives power in a non-contact manner from the non-contact power supply that supplies power in a non-contact manner so as to supply power to the battery 138. That is, the non-contact power receiver 137 is used together with the non-contact power supply to receive power by so-called wireless power supply (wireless power supply). As a method of this wireless power supply, various existing methods such as an electromagnetic induction method, a magnetic field resonance method, an electric field coupling method, a microwave method, or a laser method can be appropriately used. In addition, the non-contact power receiver 137 supplies the received power to the battery 138.

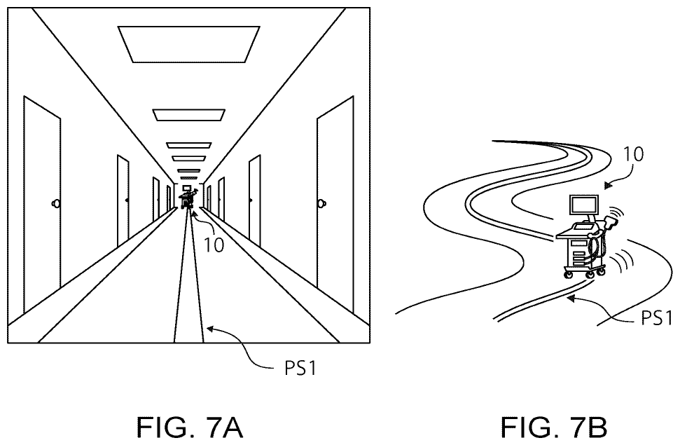

The non-contact power supply is provided, for example, on a wall, a floor, or the like of the hospital so as to be able to supply power to the non-contact power receiver 137 while the medical inspection device 10 is moving to the destination. FIGS. 7A and 7B are diagrams illustrating an example of the non-contact power supply provided in the hospital in the first embodiment. As illustrated in FIG. 7A, a non-contact power supply PS1 is provided on a floor surface of a corridor in the hospital. Then, as illustrated in FIG. 7B, the medical inspection device 10 moves along the corridor in the hospital, and thus, the non-contact power supply PS1 supplies power to the non-contact power receiver 137.

The battery 138 is a rechargeable secondary battery. The battery 138 is connected to the non-contact power receiver 137 so as to be charged by the power received by the non-contact power receiver 137. The power charged in the battery 138 may be used not only for self-traveling of the medical inspection device 10 but also for operating of the medical inspection device 10 when the inspection such as the ultrasonic inspection is performed. That is, the battery 138 may be capable of not only charging power necessary for self-traveling but also charging power necessary for the inspection such as the ultrasonic inspection

The drive unit 139 drives the support member 19 and the movable unit 21 under the control of the processing circuitry 140. The drive unit 139 includes, for example, a motor, an actuator, and the like.

The processing circuitry 140 is a control circuitry that performs overall control of the medical inspection device 10. In addition, the processing circuitry 140 is an arithmetic circuitry that performs various arithmetic operations, and includes, for example, a processor such as a CPU or a GPU. For example, the processing circuitry 140 according to the present embodiment generates an ultrasonic image from data generated by the B-mode processing circuitry 132 and the Doppler processing circuitry 133, and transmits medical data including the ultrasonic image generated via the communication interface 135 to the medical data server 20.

Thus, the processing circuitry 140 according to the present embodiment has an image generation function 141, a data transmission function 142, a diagnosis function 143, a setting data acquisition function 144, and a control function 145. The image generation function 141 corresponds to an image generation unit according to the present embodiment, the data transmission function 142 corresponds to a data transmission unit, the diagnosis function 143 corresponds to a diagnosis unit according to the present embodiment, the setting data acquisition function 144 corresponds to a setting data acquisition unit according to the present embodiment, and the control function 145 corresponds to a control unit according to the present embodiment.

In the embodiment illustrated in FIG. 6, processing functions performed by the image generation function 141, the data transmission function 142, the diagnosis function 143, the setting data acquisition function 144, and the control function 145 are stored in the memory 134 in the form of a program executable by a computer. The processing circuitry 140 is a processor that realizes a function corresponding to each program by reading and executing the program from the memory 134. In other words, the processing circuitry 140 in a state of reading each program has each function illustrated in the processing circuitry 140 of FIG. 6. Note that, in FIG. 6, although it has been described that the image generation function 141, the data transmission function 142, the diagnosis function 143, the setting data acquisition function 144, and the control function 145 are realized by the single processing circuitry 140, these functions may be realized by combining a plurality of independent processors to constitute the processing circuitry 140 and executing a program by each processor.

The image generation function 141 generates the ultrasonic image from the data generated by the B-mode processing circuitry 132 and the Doppler processing circuitry 133. That is, the image generation function 141 generates the ultrasonic image in which the intensity of the reflected wave is represented by luminance from the two-dimensional B-mode data generated by the B-mode processing circuitry 132. In addition, the image generation function 141 generates an ultrasonic image representing moving body information from the two-dimensional Doppler data generated by the Doppler processing circuitry 133. The ultrasonic image based on the Doppler data is speed image data, distributed image data, power image data, or image data obtained by combining these pieces of data.

Here, the image generation function 141 generally converts (scan converts) a scanning line signal sequence of ultrasonic scanning into a scanning line signal sequence of a video format represented by a television or the like, and generates a display ultrasonic image. Specifically, the image generation function 141 generates the display ultrasonic image by performing coordinate conversion in accordance with a scanning form of the ultrasonic wave by the ultrasonic probe 11. In addition, for example, the image generation function 141 performs, as various kinds of image processing other than the scan conversion, image processing (smoothing processing) of regenerating an average value image of luminance by using a plurality of image frames after scan conversion, image processing (edge enhancement processing) using a differential filter in the image, and the like. In addition, the image generation function 141 synthesizes character information, graduations, body marks, and the like of various parameters with the ultrasonic image.

Further, the image generation function 141 generates three-dimensional B-mode image data by performing coordinate conversion on the three-dimensional B-mode data generated by the B-mode processing circuitry 132. In addition, the image generation function 141 generates three-dimensional Doppler image data by performing coordinate conversion on the three-dimensional Doppler data generated by the Doppler processing circuitry 133. Further, the image generation function 141 can perform rendering processing on volume data in order to generate various two-dimensional images for displaying the three-dimensional image data (volume data) on the display 15.

The data transmission function 142 transmits the medical data collected in the inspection to the medical data server 20. In addition, the data transmission function 142 transmits the setting data such as the preset data and the protocol data used in the inspection to the setting data server 30. The data transmission function 142 according to the present embodiment transmits the medical data collected in the inspection and the setting data used in the inspection to the medical data server 20 and the setting data server 30 while moving to the destination.

The diagnosis function 143 diagnoses an operating state of the apparatus body 13 and/or the ultrasonic probe 11 electrically connected to the apparatus body 13. The diagnosis function 143 according to the present embodiment diagnoses the operating state of the apparatus body 13 and/or the ultrasonic probe 11 electrically connected to the apparatus body 13 while moving to the destination. For example, the diagnosis function 143 divides the apparatus body 13 into a front end portion and a back end portion, and gives an inspection input signal to each portion. Then, the diagnosis function 143 compares an initial value of an output signal stored in the memory 134 with a value of the output signal with respect to an inspection input signal to diagnose the operating state of the apparatus body 13. Note that, the diagnosis function 143 may diagnose the operating state of the apparatus body 13 in more detail for each processing block or each ASIC.

In addition, the diagnosis function 143 diagnoses the ultrasonic probe 11 including the piezoelectric vibrator. For example, the diagnosis function 143 compares an initial value of each of the plurality of piezoelectric vibrators with a feature value of the reflected wave signal based on a feature value of the reflected wave signal from the air to diagnose operating states of the plurality of piezoelectric vibrators. For example, an amplitude, a center frequency, or a bandwidth can be appropriately used as the feature value. For example, in the case of the amplitude, a sensitivity peak value is used as the feature value. Specifically, a maximum amplitude value (Vp−p) of a first reflected wave is used as the sensitivity peak value.

Then, as the diagnosis result of the operating state, the diagnosis function 143 transmits a difference between the initial value of the output signal in the apparatus body 13 and the value of the output signal with respect to the inspection input signal and/or a difference between the initial value of each of the plurality of piezoelectric vibrators in the ultrasonic probe 11 and the feature value of the reflected wave signal to the management apparatus 40 via the communication interface 135. As described above, the diagnosis function 143 diagnoses the operating state of the apparatus body 13 and/or the ultrasonic probe 11 electrically connected to the apparatus body 13, and thus, it is possible to check whether the operating state of the apparatus body 13 and the operating state of the ultrasonic probe 11 can be reproduced as the operating states of the apparatus body 13 and the ultrasonic probe 11 at the time of shipment.

The setting data acquisition function 144 acquires the setting data from the setting data server 30 based on setting data information transmitted from the management apparatus 40. Here, the setting data information is information regarding the setting data used in the inspection. The setting data information includes, for example, information indicating a location of the setting data. The setting data acquisition function 144 according to the present embodiment acquires the setting data from the setting data server 30 while moving to the destination.

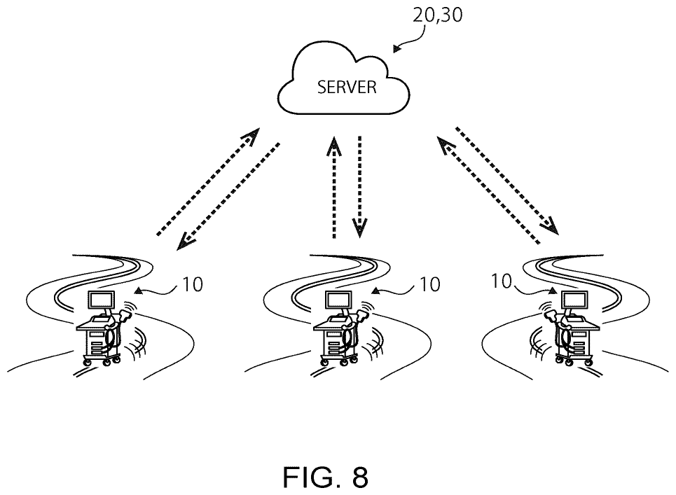

FIG. 8 is a diagram illustrating an example of the transmission and reception of the medical data and the setting data in the first embodiment. As illustrated in FIG. 8, in the plurality of medical inspection devices 10, the data transmission function 142 of each of the plurality of medical inspection devices 10 transmits the medical data collected in the inspection and the setting data used in the inspection to the servers of the medical data server 20 and the setting data server 30 while moving to the destination. In addition, in the plurality of medical inspection devices 10, the setting data acquisition function 144 of each of the plurality of medical inspection devices 10 acquires setting data of a next inspection while moving to the destination from the setting data server 30 based on the setting data information. As described above, the medical inspection device 10 transmits the medical data and the setting data and acquires the setting data while moving to the destination, and thus, the user can start the next inspection when the medical inspection device 10 arrives at the destination.

The control function 145 controls the drive unit 139 to control the support member 19. Specifically, at the destination, the control function 145 controls the support member 19 based on user information regarding a user who uses the ultrasonic diagnosis apparatus which is the medical inspection device 10, and moves the display 15 and the input interface 17 to a use position which is a position where the user uses the display 15 and the input interface 17. In addition, while moving to the destination, the control function 145 controls the support member 19 to move the display 15 and the input interface 17 to an unused position below the use position. Here, the user information is information regarding the user who uses the ultrasonic diagnosis apparatus in the inspection. For example, the user information includes information regarding a height of the user and a sitting height of the user. The user information is managed by the management apparatus 40, the hospital information system, or the like. In addition, the information regarding the control for moving the display 15 and the input interface 17 to the unused position is information regarding the amount of control of the support member 19. The information regarding the control for moving the display 15 and the input interface 17 to the unused position is stored in the memory 134, for example.

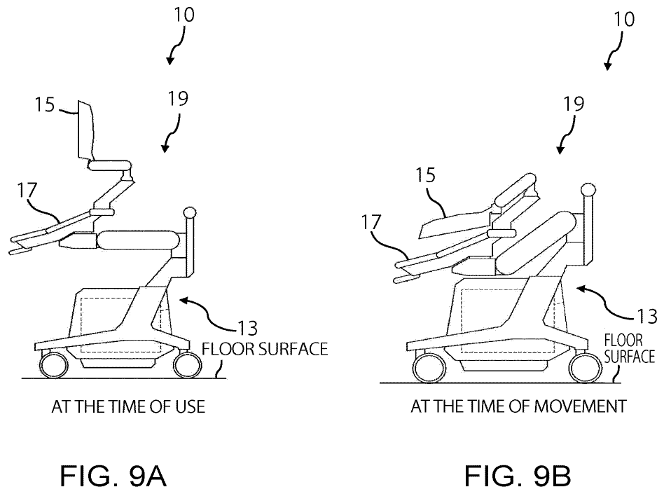

FIGS. 9A and 9B are schematic views illustrating an appearance at the time of use (FIG. 9A) and at the time of movement (FIG. 9B) of the medical inspection device 10 according to the first embodiment. As illustrated in FIG. 9A, at the time of use at the destination, the medical inspection device 10 controls the support member 19 based on the user information, to move the display 15 and the input interface 17 to the use position used by the user. Then, as illustrated in FIG. 9B, while moving to the destination, the medical inspection device 10 controls the support member 19 to move the display 15 and the input interface 17 to the unused position below the use position such that a size of the medical inspection device 10 becomes small. In addition, in the example illustrated in FIG. 9B, the medical inspection device 10 according to the present embodiment controls the support member 19 to fold the display 15 so as to overlap the input interface 17 side such that the size of the medical inspection device 10 becomes smaller. As described above, the medical inspection device 10 controls the support member 19 to move the display 15 and the input interface 17 at the time of use and at the time of movement. As a result, the medical inspection device 10 can move the display 15 and the input interface 17 to an easily viewable position or an easily operable position of the medical image of the user at the time of use, and can reduce a possibility of coming into contact with an article or a person in the hospital at the time of movement.

In addition, the control function 145 controls the drive unit 139 to control the movable unit 21. Specifically, the control function 145 receives destination information from the management apparatus 40, and controls the movable unit 21 based on the destination information. That is, the control function 145 controls the movable unit 21 to perform automatic traveling until the medical inspection device 10 arrives at the destination. As a method of this automatic traveling, an electromagnetic induction method for performing guidance by using a metal wire through which a current flows installed on the floor surface may be used, an optical induction method for performing guidance by using an induction wire drawn on the floor surface may be used, a magnetic induction method for performing guidance by detecting a magnetic field from a floor by using a magnetic body installed on the floor surface by a sensor may be used, an image recognition method for performing guidance by reading an image such as a QR code (registered trademark) or an AR marker disposed on the floor surface or a ceiling surface with a camera may be used, a laser method for installing a reflection plate on a wall or a column and estimating a position by reflection of a laser may be used, and various existing methods such as a simultaneous localization and mapping (SLAM) method for estimating a position by using a sensor such as a camera or a laser, and an encoder or a gyro sensor may be appropriately used. In the present embodiment, the control function 145 and the movable unit 21 constitute the self-traveling function.

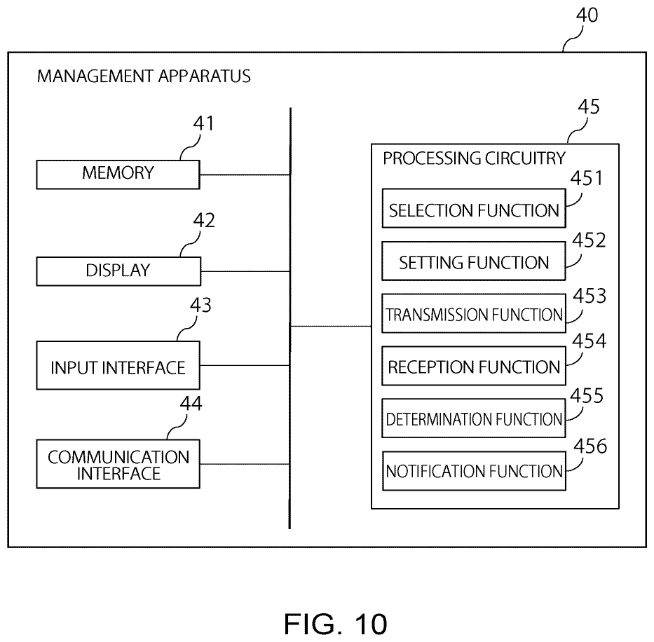

FIG. 10 is a block diagram illustrating an example of a configuration of the management apparatus 40 according to the first embodiment. As illustrated in FIG. 10, the management apparatus 40 includes a memory 41, a display 42, an input interface 43, a communication interface 44, and a processing circuitry 45. Note that, in the example illustrated in FIG. 10, although the management apparatus 40 includes the display 42 and the input interface 43, the management apparatus 40 may not include the display 42 and the input interface 43.

The memory 41 stores information regarding the medical inspection device 10 present in the hospital. The information regarding the medical inspection device 10 includes, for example, information such as a type and a model of the medical inspection device 10 and operating status information regarding an operating status of the medical inspection device 10. Here, the operating status information is information regarding the operating status of the medical inspection device 10, and includes, for example, a cumulative operating time of the medical inspection device 10, an operating rate of the medical inspection device 10, and the like. In addition, the memory 41 stores information regarding the medical inspection fixture present in the hospital. In addition, the memory 41 stores the inspection and the information regarding the medical inspection fixture necessary in accordance with the inspection in association with each other. In addition, the memory 41 stores the inspection order transmitted from the terminal apparatus 50. In addition, the memory 41 stores a threshold for the difference between the initial value of the output signal in the apparatus body 13 and the value of the output signal with respect to the inspection input signal, a threshold for the difference between the initial value of each of the plurality of piezoelectric vibrators in the ultrasonic probe 11 and the feature value of the reflected wave signal, and the like.

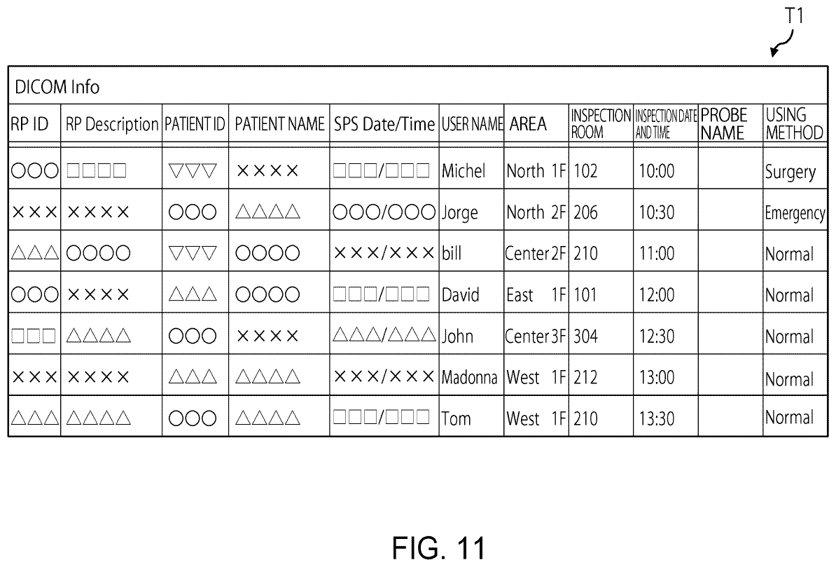

In addition, the memory 41 stores an inspection reservation table for each medical inspection device 10. The inspection reservation table is a table related to the inspection reservation, and is associated with information such as a user name, a use place, and a use start time of the medical inspection device 10. FIG. 11 is a diagram illustrating an example of the inspection reservation table stored in the memory 41 of the management apparatus 40 according to the first embodiment. As illustrated in FIG. 11, an inspection reservation table T1 according to the present embodiment stores a patient ID, a patient name, a user name, an area, an inspection room, a use start time, a probe name, a use method, and the like in association with one another. For example, when “patient ID: 64041985” is used as an example, the medical inspection device 10 according to the inspection reservation table T1 is reserved from “10:00” in an inspection room “102” of “North 1F” by a user name “Michel”. In the example illustrated in FIG. 11, the inspection reservation table T1 is stored in a DICOM format.

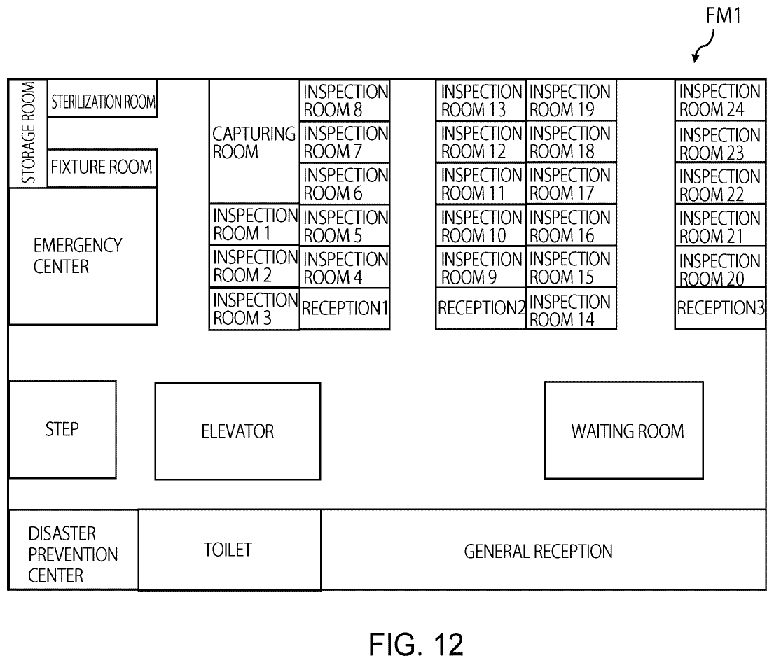

Further, the memory 41 stores a floor map in the hospital. FIG. 12 is a diagram illustrating an example of the floor map stored in the memory 41 of the management apparatus 40 according to the first embodiment. As illustrated in FIG. 12, a floor map FM1 according to the present embodiment includes a medical inspection room corresponding to the inspection room, an emergency center corresponding to the emergency room, a sterilization room, a fixture room, a storage room in which unused medical inspection devices 10 are stored, and the like.

The display 42 is a display apparatus that displays various kinds of information. For example, the display 42 displays the inspection order stored in the memory 41 and the floor map FM1 on which the positional information of the medical inspection device 10 is displayed. In the present embodiment, the display 42 includes, for example, a liquid crystal display, a cathode ray tube (CRT) display, or the like.

The input interface 43 is an input apparatus for performing various settings and the like. The input interface 43 is realized by, for example, a trackball, a switch button, a mouse, a keyboard, a touch pad that performs an input operation by touching an operation surface, a touch monitor in which a display screen and the touch pad are integrated, a non-contact input circuitry using an optical sensor, a sound input circuitry, and the like. The input interface 43 is connected to the processing circuitry 45, converts an input operation accepted from an operator into an electric signal, and outputs the electric signal to the processing circuitry 45. Note that, in the present specification, the input interface 43 is not limited to the interface including physical operation components such as a mouse and a keyboard. For example, an electric signal processing circuitry that accepts an electric signal corresponding to an input operation from an external input device provided separately from the apparatus and outputs the electric signal to the processing circuitry 45 is also included in the example of the input interface 43.

The communication interface 44 is connected to the medical inspection device 10, the medical data server 20, the setting data server 30, and the terminal apparatus 50 via, for example, the in-hospital network NW1, and performs data communication with the medical inspection device 10, the medical data server 20, the setting data server 30, and the terminal apparatus 50.

The processing circuitry 45 is a circuitry that performs overall control of the management apparatus 40. In addition, the processing circuitry 45 is an arithmetic circuitry that performs various arithmetic operations, and includes, for example, a processor such as a CPU or a GPU. For example, the processing circuitry 45 according to the present embodiment selects an available medical inspection device 10 from among one or the plurality of medical inspection devices 10, sets the destination of the medical inspection device 10, and transmits the destination information regarding the destination to the medical inspection device 10.

Thus, the processing circuitry 45 according to the present embodiment has a selection function 451, a setting function 452, a transmission function 453, a reception function 454, a determination function 455, and a notification function 456. The selection function 451 corresponds to a selection unit according to the present embodiment, the setting function 452 corresponds to a setting unit according to the present embodiment, the transmission function 453 corresponds to a transmission unit according to the present embodiment, the reception function 454 corresponds to a reception unit according to the present embodiment, the determination function 455 corresponds to a determination unit according to the present embodiment, and the notification function 456 corresponds to a notification unit according to the present embodiment.

In the embodiment illustrated in FIG. 10, processing functions performed by the selection function 451, the setting function 452, the transmission function 453, the reception function 454, the determination function 455, and the notification function 456 are stored in the memory 41 in the form of a program executable by a computer. The processing circuitry 45 is a processor that realizes a function corresponding to each program by reading and executing the program from the memory 41. In other words, the processing circuitry 45 in a state of reading each program has each function illustrated in the processing circuitry 45 of FIG. 10. Note that, in FIG. 10, although it has been described that the selection function 451, the setting function 452, the transmission function 453, the reception function 454, the determination function 455, and the notification function 456 are realized by the single processing circuitry 45, these functions may be realized by combining a plurality of independent processors to constitute the processing circuitry 45 and executing a program by each processor.

The selection function 451 selects the available medical inspection device 10 based on the inspection order transmitted from the terminal apparatus 50. In addition, the selection function 451 selects a medical inspection fixture necessary for inspection from among a plurality of medical inspection fixtures based on the inspection order. Further, the selection function 451 selects setting data necessary for the inspection from among a plurality of pieces of setting data based on the inspection order.

The setting function 452 sets the destination of the medical inspection device 10 selected by the selection function 451. The destination is, for example, the inspection room, the emergency room, the hospital room, or the like.

The transmission function 453 transmits the destination information to the medical inspection device 10 selected by the selection function 451. Here, the destination information is information regarding the destination, and includes, for example, positional information of the destination and information such as a moving route to the destination. In addition, the transmission function 453 transmits medical inspection fixture information to the medical inspection device 10. Here, the medical inspection fixture information is information regarding the medical inspection fixture selected by the selection function 451, and includes, for example, information such as a name of the medical inspection fixture and a type of the medical inspection fixture. Further, the transmission function 453 transmits the setting data information to the medical inspection device 10.

The reception function 454 receives a diagnosis result of the operating state diagnosed by the diagnosis function 143 of the medical inspection device 10 from the medical inspection device 10. The determination function 455 determines the operating state based on the diagnosis result. In a case where the medical inspection device 10 arrives at the destination, the notification function 456 notifies that the medical inspection device 10 arrives at the destination.

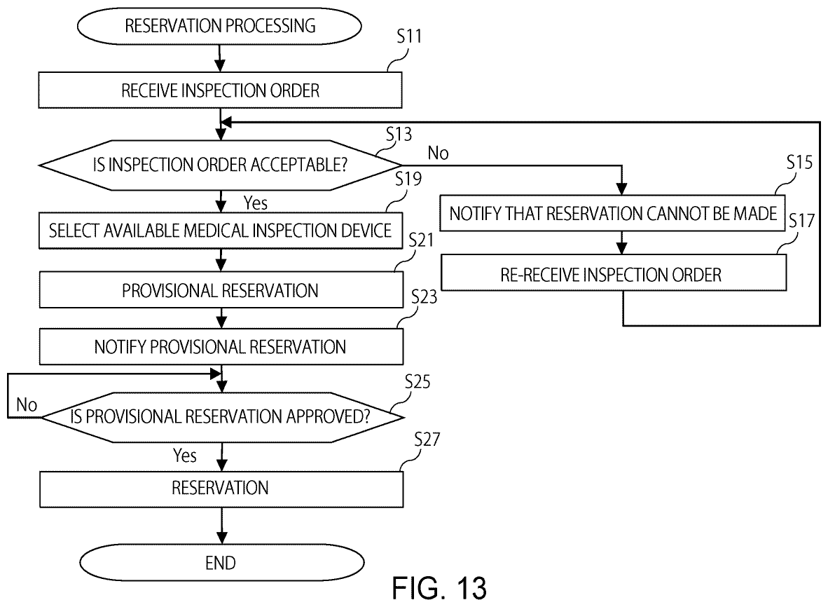

FIG. 13 is a flowchart for describing contents of reservation processing executed by the management apparatus 40 according to the first embodiment. In this reservation processing, the inspection order is received, the available medical inspection device 10 is selected, and the inspection order is reserved. For example, this reservation processing is processing executed in a case where the management apparatus 40 receives the inspection order from the terminal apparatus 50.

As illustrated in FIG. 13, first, the selection function 451 realized by the processing circuitry 45 of the management apparatus 40 receives the inspection order from the terminal apparatus 50 via the communication interface 44 of the management apparatus 40 (step S11). Specifically, the selection function 451 receives the inspection order including at least the medical inspection device information and the inspection date and time from the terminal apparatus 50.

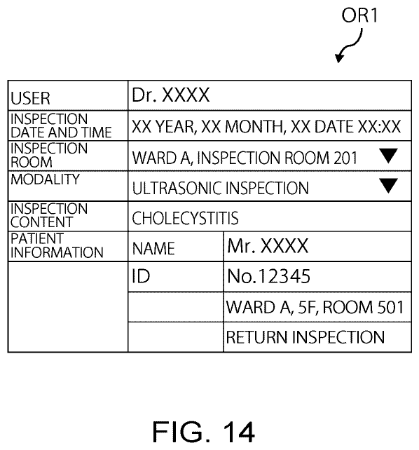

FIG. 14 is a diagram illustrating an example of the inspection order received by the management apparatus 40 according to the first embodiment. In the example illustrated in FIG. 14, an inspection order OR1 includes the user corresponding to the user name, the inspection date and time, the inspection room corresponding to the inspection place, the modality, the inspection content, the patient information including the patient name and the patient ID, information for identifying whether the patient is the outpatient or the inpatient, and the inspection number information. In the example illustrated in FIG. 14, the modality corresponds to the medical inspection device information according to the present embodiment.

Subsequently, as illustrated in FIG. 13, the selection function 451 realized by the processing circuitry 45 of the management apparatus 40 determines whether or not the inspection order OR1 is acceptable (step S13). Specifically, the selection function 451 refers to the inspection reservation table T1 of each of the medical inspection devices 10 in the medical inspection device information stored in the memory 41, and determines whether or not the inspection order OR1 received in step S11 is acceptable at the inspection date and time included in the inspection order OR1.

Then, in step S13, in a case where the inspection order OR1 is not acceptable (step S13: No), the selection function 451 realized by the processing circuitry 45 of the management apparatus 40 notifies the user that the reservation cannot be made (step S15). Specifically, the selection function 451 notifies the user that the reservation cannot be made by transmitting information indicating that the reservation cannot be made to the terminal apparatus 50 via the communication interface 44. Then, the user who has received the notification that the reservation cannot be made changes the inspection date and time in the inspection order OR1.

Subsequently, as illustrated in FIG. 13, the selection function 451 realized by the processing circuitry 45 of the management apparatus 40 re-receives the inspection order OR1 (step S17). Specifically, the selection function 451 re-receives the inspection order OR1 whose inspection date and time is changed from the terminal apparatus 50. Then, the selection function 451 returns to step S13 and determines whether or not the inspection order OR1 whose inspection date and time is changed is acceptable. That is, the processing of steps S13 to S17 is repeated until the inspection order becomes acceptable.

On the other hand, in step S13, in a case where the inspection order OR1 is acceptable (step S13: Yes), the selection function 451 realized by the processing circuitry 45 of the management apparatus 40 selects the available medical inspection device based on the medical inspection device information and the inspection date and time (step S19). Specifically, the selection function 451 refers to the inspection reservation table T1 of each of the medical inspection devices 10 stored in the memory 41, and selects the available medical inspection device 10 at the inspection date and time included in the inspection order OR1. In step S19, in a case where there is a plurality of available medical inspection devices 10, the selection function 451 refers to the operating status information regarding to the operating status of each of the plurality of available medical inspection devices 10, and selects the medical inspection device 10 based on the operating status information. For example, the selection function 451 selects the medical inspection device 10 having a low operating rate or the medical inspection device 10 having a small cumulative operating time based on the operating status information. As described above, the selection function 451 selects the medical inspection device 10 in accordance with the operating status, and thus, it is possible to evenly operate the medical inspection devices 10 present in the hospital without deviating a use frequency of each of the medical inspection devices 10.

Subsequently, as illustrated in FIG. 13, the selection function 451 realized by the processing circuitry 45 of the management apparatus 40 provisionally reserves the selected medical inspection device 10 (step S21). Specifically, the selection function 451 provisionally reserves the medical inspection device 10 by provisionally registering the inspection order OR1 in the inspection reservation table T1 of the medical inspection device 10 selected in step S19.

Subsequently, as illustrated in FIG. 13, the selection function 451 realized by the processing circuitry 45 of the management apparatus 40 notifies the provisional reservation of the medical inspection device 10 (step S23). Specifically, the selection function 451 notifies the terminal apparatus 50 of the provisional reservation of the medical inspection device 10 via the communication interface 44.

Subsequently, as illustrated in FIG. 13, the selection function 451 realized by the processing circuitry 45 of the management apparatus 40 determines whether or not the provisional reservation is approved (step S25). Specifically, the selection function 451 determines whether or not the provisional reservation is approved by determining whether or not a signal for approving the provisional reservation is received from the terminal apparatus 50 via the communication interface 44. Then, in step S25, in a case where the provisional reservation is not approved (step S25: No), the selection function 451 repeats step S25 and stands by until the provisional reservation is approved.

On the other hand, in a case where the provisional reservation is approved in step S25 (step S25: Yes), the selection function 451 realized by the processing circuitry 45 of the management apparatus 40 reserves the medical inspection device 10 (step S27). Specifically, the selection function 451 reserves the medical inspection device 10 by registering the inspection order OR1 in the inspection reservation table T1 of the medical inspection device 10 selected in step S19.

In step S27, the medical inspection device 10 is reserved, and thus, the reservation processing according to the present embodiment is ended.

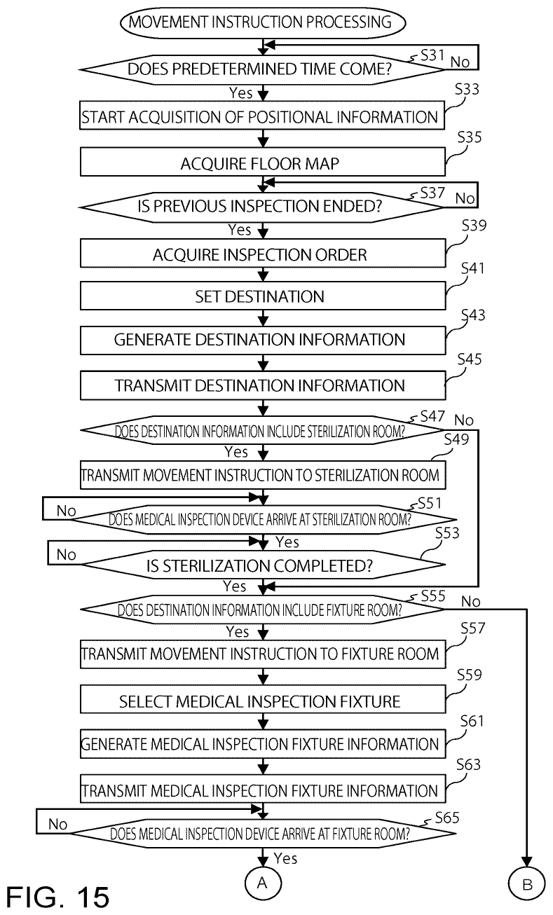

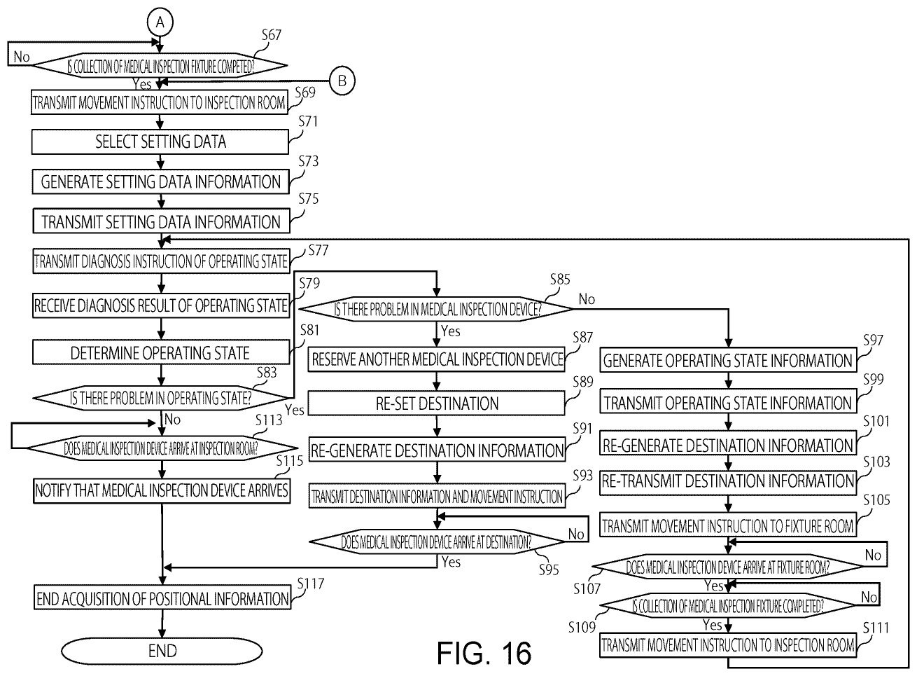

FIGS. 15 and 16 are flowcharts for describing an example of movement instruction processing executed in the management apparatus 40 according to the first embodiment. In this destination transmission processing, the management apparatus 40 sets the destination of the medical inspection device 10, generates the destination information based on the positional information of the medical inspection device 10 and the destination of the medical inspection device 10, transmits the destination information to the medical inspection device 10, and transmits a movement instruction to the medical inspection device 10. For example, this movement instruction processing is processing executed in a case where a predetermined time comes. In addition, this movement instruction processing is processing executed for each medical inspection device 10 managed by the management apparatus 40. In the following description, description will be given focusing on one medical inspection device 10 among the medical inspection devices 10 managed by the management apparatus 40.

As illustrated in FIG. 15, first, the setting function 452 realized by the processing circuitry 45 of the management apparatus 40 determines whether or not the predetermined time comes (step S31). Specifically, the setting function 452 refers to the inspection reservation table T1 stored in the memory 41 of the management apparatus 40 and determines whether or not the predetermined time comes. Here, the predetermined time is a time several minutes before the inspection date and time included in the inspection reservation table T1 or the inspection date and time included in the inspection reservation table T1.

Subsequently, as illustrated in FIG. 15, the setting function 452 realized by the processing circuitry 45 of the management apparatus 40 starts the acquisition of the positional information (step S33). Specifically, the setting function 452 starts the acquisition of the positional information by acquiring the positional information of the medical inspection device 10 from the position sensor 136 of the medical inspection device 10 via the communication interface 44.

Subsequently, as illustrated in FIG. 15, the setting function 452 realized by the processing circuitry 45 of the management apparatus 40 acquires the floor map FM1 (step S35). Specifically, the setting function 452 acquires the floor map FM1 from the memory 41 of the management apparatus 40. Then, the setting function 452 displays the position of the medical inspection device 10 on the floor map FM1 by reflecting the positional information of the medical inspection device 10 on the acquired floor map FM1. Note that, in a case where positional information of another medical inspection device 10 managed by the management apparatus 40 is acquired in step S33, the position of the other medical inspection device 10 is also displayed on the floor map.

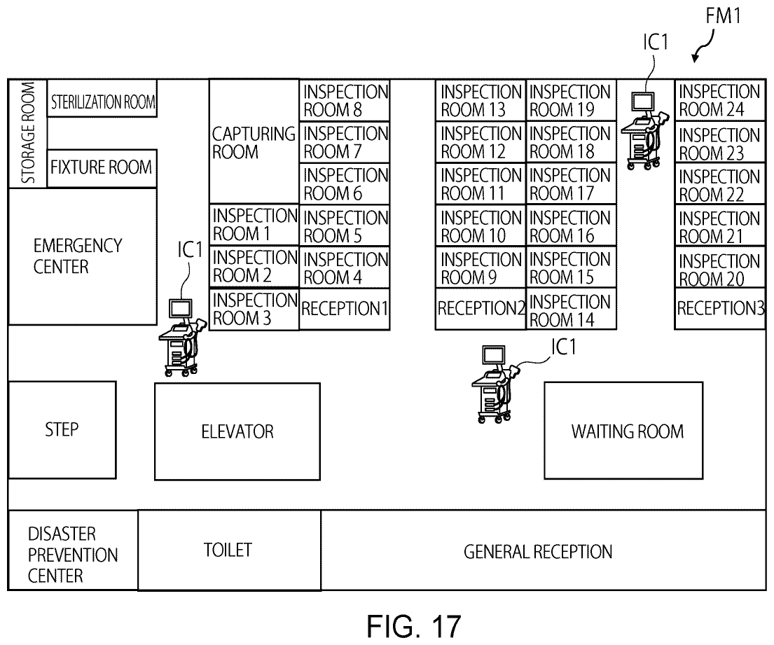

FIG. 17 is a diagram illustrating an example of the floor map FM1 on which the position of the medical inspection device 10 is displayed in the first embodiment. The floor map FM1 illustrated in FIG. 17 represents a hospital facility. On the floor map FM1, the position of the medical inspection device 10 is displayed by an icon IC1 that simulates a shape of the medical inspection device 10.

Subsequently, as illustrated in FIG. 15, the setting function 452 realized by the processing circuitry 45 of the management apparatus 40 determines whether or not the previous inspection is ended (step S37). Specifically, the setting function 452 determines whether or not the previous inspection of the medical inspection device 10 is ended based on the floor map FM1 and the positional information of the medical inspection device 10. More specifically, when the inspection is ended, since the medical inspection device 10 is taken out to the corridor by the medical worker, the setting function 452 determines whether or not the previous inspection is ended by determining whether or not the medical inspection device 10 moves from the inspection room to the corridor based on the floor map FM1 reflecting the positional information of the medical inspection device 10. Then, in step S37, in a case where the previous inspection is not ended (step S37: No), the setting function 452 repeats the processing of step S37 and stands by. That is, the management apparatus 40 stands by until the previous inspection is ended.

On the other hand, in step S37, in a case where the previous inspection is ended (step S37: Yes), the setting function 452 realized by the processing circuitry 45 of the management apparatus 40 acquires the inspection order OR1 (step S39). Specifically, the setting function 452 acquires the inspection order OR1 whose inspection date and time arrives from the memory 41 of the management apparatus 40.

Subsequently, as illustrated in FIG. 15, the setting function 452 realized by the processing circuitry 45 of the management apparatus 40 sets the destination (step S41). Specifically, the setting function 452 sets the destination of the medical inspection device 10 based on the inspection order OR1. More specifically, in a case where the setting function 452 acquires the inspection order OR1 illustrated in FIG. 14 in step S39, the setting function 452 sets “ward A and inspection room 201” as the destination in step S41. Note that, in step S41, although the setting function 452 sets the destination of the medical inspection device 10 based on the inspection order OR1, the setting function 452 may set the destination of the medical inspection device 10 by referring to the inspection reservation table T1 instead of the inspection order OR1.

Subsequently, as illustrated in FIG. 15, the setting function 452 realized by the processing circuitry 45 of the management apparatus 40 generates the destination information (step S43). Specifically, the setting function 452 generates the destination information based on the inspection order OR1 acquired in step S39, the positional information of the medical inspection device 10, the floor map FM1 acquired in step S35, and the destination set in step S41. In the present embodiment, the setting function 452 generates, as the destination information, a movement plan until moving to the destination of the medical inspection device 10. For example, in a case where the inspection is performed before this inspection, the setting function 452 generates, as the destination information, the movement plan of the medical inspection device 10 until moving to the inspection room via the sterilization room and the fixture room.

Note that, although the movement plan of the medical inspection device 10 described above includes the sterilization room and the fixture room, the movement plan of the medical inspection device 10 does not necessarily include the sterilization room and the fixture room. That is, in the movement plan of the medical inspection device 10, the medical inspection device arbitrary passes through the sterilization room and the fixture room. For example, in a case where the medical inspection device 10 is used for the first time on an inspection date, and in a case where the inspection is not performed before the inspection, the sterilization room may not be included in the movement plan of the medical inspection device 10. In a case where the same inspection as the previous inspection is performed, the fixture room may not be included in the movement plan of the medical inspection device 10.

Subsequently, as illustrated in FIG. 15, the transmission function 453 realized by the processing circuitry 45 of the management apparatus 40 transmits the destination information (step S45). Specifically, the transmission function 453 transmits the destination information to the medical inspection device 10 via the communication interface 44.

Subsequently, as illustrated in FIG. 15, the setting function 452 realized by the processing circuitry 45 of the management apparatus 40 determines whether or not the destination information includes the sterilization room (step S47). Specifically, the setting function 452 determines whether or not the destination information generated in step S43 includes the sterilization room.

Then, in step S47, in a case where the destination information includes the sterilization room (step S47: Yes), the setting function 452 realized by the processing circuitry 45 of the management apparatus 40 transmits the movement instruction to the sterilization room to the medical inspection device 10 (step S49). As a result, the management apparatus 40 starts moving the medical inspection device 10 to the sterilization room.

Subsequently, as illustrated in FIG. 15, the setting function 452 realized by the processing circuitry 45 of the management apparatus 40 determines whether or not the medical inspection device 10 arrives at the sterilization room (step S51). Specifically, the setting function 452 determines whether or not the positional information of the medical inspection device 10 arrives at the sterilization room by determining whether or not the positional information of the medical inspection device 10 is the position of the sterilization room on the floor map FM1 based on the positional information acquired from the medical inspection device 10 and the floor map FM1. Then, in step S51, in a case where the medical inspection device 10 does not arrive at the sterilization room (step S51: No), the setting function 452 repeats step S51 and stands by.

On the other hand, in step S51, in a case where the medical inspection device 10 arrives at the sterilization room (step S51: Yes), the setting function 452 realized by the processing circuitry 45 of the management apparatus 40 determines whether or not sterilization is completed (step S53). Specifically, the setting function 452 determines whether or not sterilization is completed by determining whether or not a notification of completion of various kinds of processing such as the sterilization or the like of the medical inspection device 10 is received from the terminal apparatus installed in the sterilization room via the communication interface 44. Then, in step S53, in a case where sterilization is not completed (step S53: No), the setting function 452 repeats step S53 and stands by.

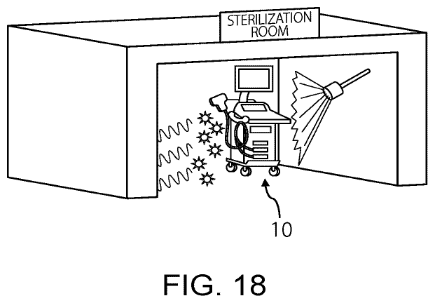

FIG. 18 is a diagram illustrating the medical inspection device 10 moved to the sterilization room in the first embodiment. In the sterilization room illustrated in FIG. 18, cleaning, washing, disinfection, fumigation and/or sterilization (hereinafter, referred to as sterilization or the like) of the medical inspection device 10 and/or the medical inspection fixture is performed. These kinds of processing such as sterilization are performed by, for example, a worker having specialized knowledge (hereinafter, referred to as a worker for sterilization or the like). Specifically, the worker for sterilization or the like cleans the display 15 and the input interface 17 of the medical inspection device 10 and the medical inspection fixture such as a heart sound sensor and a pulse wave sensor by using, for example, a neutral detergent or ethanol for disinfection. In addition, the worker for sterilization or the like cleans the medical inspection device and/or the medical inspection fixture by using, for example, an enzyme washing agent. In addition, the worker for sterilization or the like disinfects or fumigates the medical inspection device and/or the medical inspection fixture by using, for example, a fumigation disinfectant such as glutaraldehyde or ortho-phthalaldehyde. In addition, the worker sterilizes the medical inspection fixture and/or the medical inspection device by using, for example, hydrogen peroxide plasma. Further, in a procedure of performing cleaning, washing, disinfection, fumigation, and/or sterilization (hereinafter, referred to as sterilization or the like) of the medical inspection device and/or the medical inspection fixture, the worker for sterilization or the like visually checks whether or not there is an abnormality in appearance in the medical inspection device 10 or the medical inspection fixture. When various kinds of processing are completed, the worker for sterilization or the like notifies the management apparatus 40 of the completion of various kinds of processing. As described above, the worker for sterilization or the like performs various kinds of processing in the sterilization room, and thus, it is possible to stop the use of the medical inspection device 10 and the medical inspection fixture having the abnormality in appearance while keeping the medical inspection device 10 and/or the medical inspection fixture clean.

On the other hand, in a case where the sterilization is completed in step S53 (step S53: Yes) or in a case where the destination information does not include the sterilization room in step S47 (step S47: No), the setting function 452 realized by the processing circuitry 45 of the management apparatus 40 determines whether or not the destination information includes the fixture room (step S55). Specifically, the setting function 452 determines whether or not the destination information generated in step S43 includes the fixture room.

Then, in step S53, in a case where the destination information includes the fixture room (step S55: Yes), the transmission function 453 realized by the processing circuitry 45 of the management apparatus 40 transmits the movement instruction to the fixture room to the medical inspection device 10 (step S57). As a result, the management apparatus 40 starts moving the medical inspection device 10 to the fixture room.

Subsequently, as illustrated in FIG. 15, the selection function 451 realized by the processing circuitry 45 of the management apparatus 40 selects the medical inspection fixture (step S59). Specifically, the selection function 451 selects the medical inspection fixture necessary for the inspection from among the medical inspection fixtures managed in the hospital by referring to the information regarding the necessary medical inspection fixture in accordance with the inspection stored in the memory 41 based on the inspection order OR1 acquired in step S39.

Subsequently, as illustrated in FIG. 15, the selection function 451 realized by the processing circuitry 45 of the management apparatus 40 generates the medical inspection fixture information (step S61). Specifically, the selection function 451 generates the medical inspection fixture information on the medical inspection fixture selected in step S59.

Subsequently, as illustrated in FIG. 15, the transmission function 453 realized by the processing circuitry 45 of the management apparatus 40 transmits the medical inspection fixture information (step S63). Specifically, the transmission function 453 transmits the medical inspection fixture information generated in step S61 to the medical inspection device 10 via the communication interface 44. Note that, in step S63, although the transmission function 453 transmits the medical inspection fixture information to the medical inspection device 10, a transmission destination of the medical inspection fixture information is not limited to the medical inspection device 10. That is, the transmission destination of the medical inspection fixture information is arbitrary, and for example, the transmission function 453 may transmit the medical inspection fixture information to the terminal apparatus or the like installed in the fixture room.

Subsequently, as illustrated in FIG. 15, the setting function 452 realized by the processing circuitry 45 of the management apparatus 40 determines whether or not the medical inspection device 10 arrives at the fixture room (step S65). Specifically, the setting function 452 determines whether or not the medical inspection device 10 arrives at the fixture room by determining whether or not the positional information of the medical inspection device 10 is the position of the fixture room on the floor map FM1 based on the positional information acquired from the medical inspection device 10 and the floor map FM1. Then, in step S65, in a case where the medical inspection device 10 does not arrive at the fixture room (step S65: No), the setting function 452 repeats step S65 and stands by.

On the other hand, in a case where the medical inspection device 10 arrives at the fixture room in step S65 (step S65: Yes), the setting function 452 realized by the processing circuitry 45 of the management apparatus 40 determines whether or not the collection of the medical inspection fixture is completed (step S67). Specifically, the setting function 452 determines whether or not the collection of the medical inspection fixture is completed by determining whether or not a notification of the completion is received from the terminal apparatus installed in the fixture room. Then, in step S67, in a case where the collection of the medical inspection fixture is not completed (step S67: No), the setting function 452 repeats step S67 and stands by.

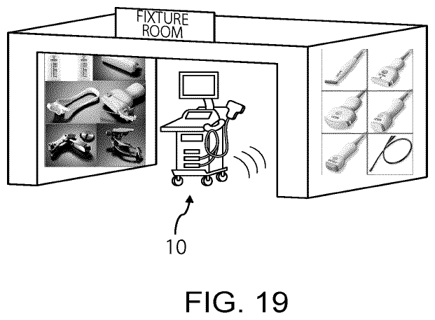

FIG. 19 is a diagram illustrating the medical inspection device 10 moved to the fixture room in the first embodiment. In the fixture room illustrated in FIG. 19, a medical inspection fixture necessary for a next inspection is collected and replaced. The collection of the medical inspection fixture is performed by, for example, a fixture replacement worker. Hereinafter, a series of flows of the collection and replacement of the medical inspection fixture will be described by using a case where the previous inspection is an abdominal inspection and the next inspection is a circulatory organ inspection. When the medical inspection device 10 arrives at the fixture room, the fixture replacement worker detaches and stores a general-purpose convex probe and/or a general-purpose linear probe used in the abdominal inspection from the apparatus body 13 of the medical inspection device 10. Then, the fixture replacement worker collects a general-purpose adult sector probe, a general-purpose convex probe, and a general-purpose linear probe used in the circulatory inspection, and connects these probes to the apparatus body 13. In addition, the fixture replacement worker removes and stores an ultrasonic jelly for the abdominal inspection, and sets an ultrasonic jelly for the circulatory organ inspection in the medical inspection device 10. In addition, the fixture replacement worker sets an ECG cable and an electrode pad in the medical inspection device 10. The electrode pad may be an ECG clip or a disposable ECG electrode pad. Further, the fixture replacement worker replaces a towel set in the medical inspection device 10 with a new towel. When these kinds of processing are completed, the fixture replacement worker notifies the management apparatus 40 of the completion of the processing.

On the other hand, in step S67, in a case where the collection of the medical inspection fixture is completed (step S67: Yes), the transmission function 453 realized by the processing circuitry 45 of the management apparatus 40 transmits the movement instruction to the inspection room (step S69). As a result, the management apparatus 40 starts moving the medical inspection device 10 to the inspection room.

Subsequently, as illustrated in FIG. 16, the selection function 451 realized by the processing circuitry 45 of the management apparatus 40 selects the setting data (step S71). Specifically, the selection function 451 selects the setting data necessary for the inspection from among the pieces of setting data stored in the setting data server 30 based on the inspection order acquired in step S39.

Subsequently, as illustrated in FIG. 16, the selection function 451 realized by the processing circuitry 45 of the management apparatus 40 generates the setting data information (step S73). Specifically, the selection function 451 generates the setting data information regarding the setting data selected in step S71.

Subsequently, as illustrated in FIG. 16, the transmission function 453 realized by the processing circuitry 45 of the management apparatus 40 transmits the setting data information (step S75). Specifically, the transmission function 453 transmits the setting data information generated in step S73 to the medical inspection device 10 via the communication interface 44. As a result, the management apparatus 40 causes the medical inspection device 10 to acquire the setting data.

Subsequently, as illustrated in FIG. 16, the transmission function 453 realized by the processing circuitry 45 of the management apparatus 40 transmits a diagnosis instruction of the operating state (step S77). As a result, the management apparatus 40 causes the diagnosis function 143 realized by the processing circuitry 140 of the medical inspection device 10 to diagnose the operating state of the apparatus body 13 and/or the ultrasonic probe 11 electrically connected to the apparatus body 13.

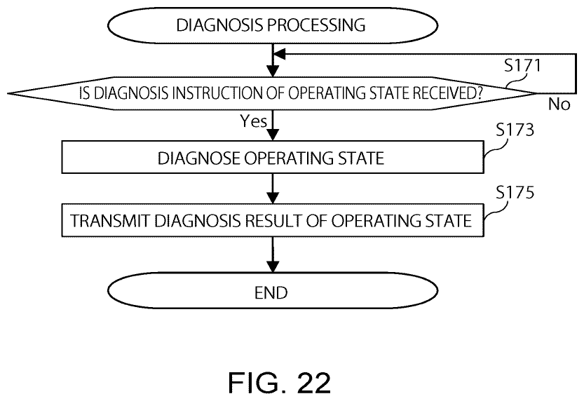

Subsequently, as illustrated in FIG. 16, the reception function 454 realized by the processing circuitry 45 of the management apparatus 40 receives the diagnosis result of the operating state (step S79). Specifically, the reception function 454 receives the diagnosis result of the operating state of the apparatus body 13 and/or the ultrasonic probe 11 electrically connected to the apparatus body 13 from the medical inspection device 10 in step S175 of FIG. 22 to be described later.

Subsequently, as illustrated in FIG. 16, the determination function 455 realized by the processing circuitry 45 of the management apparatus 40 determines the operating state (step S81). Specifically, the determination function 455 determines the operating state based on the diagnosis result of the operating state received in step S77. More specifically, for example, in a case where the difference between the initial value of the output signal in the apparatus body 13 and the value of the output signal with respect to the inspection input signal is received as the diagnosis result of the operating state in step S79, the determination function 455 determines the operating state of the medical inspection device 10 by determining whether or not the difference between the initial value of the output signal in the apparatus body 13 and the value of the output signal with respect to the inspection input signal exceeds a predetermined threshold stored in the memory 41.

Subsequently, as illustrated in FIG. 16, the determination function 455 realized by the processing circuitry 45 of the management apparatus 40 determines whether or not there is a problem in the operating state (step S83). Specifically, the determination function 455 determines whether or not there is the problem in the operating state of the apparatus body 13 and/or the ultrasonic probe 11 electrically connected to the apparatus body 13 based on the determination result of the operating state in step S79.

Then, in step S83, in a case where there is the problem in the operating state (step S83: Yes), the determination function 455 realized by the processing circuitry 45 of the management apparatus 40 determines whether or not there is a problem in the medical inspection device 10 (step S85). Specifically, the determination function 455 determines whether or not there is the problem in the operating state of the apparatus body 13 based on the determination result of the operating state in step S81.

Then, in step S85, in a case where there is the problem in the medical inspection device 10 (step S85: Yes), the determination function 455 realized by the processing circuitry 45 of the management apparatus 40 reserves another medical inspection device 10 (step S87). Specifically, the determination function 455 selects another available medical inspection device 10 based on the inspection order OR1, and reserves the other medical inspection device 10.

Subsequently, as illustrated in FIG. 16, the setting function 452 realized by the processing circuitry 45 of the management apparatus 40 re-sets the destination (step S89). Specifically, the destination is re-set in order to move the medical inspection device 10 having the problem in the operating state. For example, the setting function 452 re-sets, as the destination, the storage room in which unused medical inspection devices 10 are stored. Note that, although the setting function 452 re-sets the storage room as the destination, the movement destination of the medical inspection device 10 having the problem in the operating state is not limited to the storage room. That is, the destination of the medical inspection device 10 having the problem in the operating state is arbitrary.

Subsequently, as illustrated in FIG. 16, the setting function 452 realized by the processing circuitry 45 of the management apparatus 40 regenerates the destination information (step S91). Specifically, the setting function 452 generates, as the destination information, the movement plan until moving to the destination based on the inspection order OR1 acquired in step S37, the positional information of the medical inspection device 10, the floor map FM1 acquired in step S35, and the destination set in step S87. More specifically, for example, the setting function 452 generates the movement plan from a current position of the medical inspection device 10 to the storage room which is the destination of the medical inspection device 10.

Subsequently, as illustrated in FIG. 16, the transmission function 453 realized by the processing circuitry 45 of the management apparatus 40 transmits the destination information and the movement instruction (step S93). Specifically, the transmission function 453 transmits the destination information and the movement instruction to the medical inspection device 10 via the communication interface 44.

Subsequently, as illustrated in FIG. 16, the setting function 452 realized by the processing circuitry 45 of the management apparatus 40 determines whether or not the medical inspection device 10 arrives at the destination (step S95). Specifically, the setting function 452 determines whether or not the medical inspection device 10 arrives at the destination by determining whether or not the positional information of the medical inspection device 10 is the destination position on the floor map FM1 based on the positional information acquired from the medical inspection device 10 and the floor map FM1. Then, in step S95, in a case where the medical inspection device 10 does not arrive at the destination (step S95: No), the setting function 452 repeats step S95 and stands by.

On the other hand, in a case where there is no problem in the medical inspection device 10 in step S85 (step S85: No), that is, in a case where there is a problem in the medical inspection fixture, the setting function 452 realized by the processing circuitry 45 of the management apparatus 40 generates the operating state information (step S97). Specifically, the setting function 452 generates the operating state information as the information regarding the medical inspection fixture having the problem in the operating state based on the determination result of the operating state.

Subsequently, as illustrated in FIG. 16, the transmission function 453 realized by the processing circuitry 45 of the management apparatus 40 transmits the operating state information (step S99). Specifically, the transmission function 453 transmits the operating state information generated in step S97 to the medical inspection device 10 via the communication interface 44. Note that, in step S99, although the transmission function 453 transmits the operating state information to the medical inspection device 10, a transmission destination of the operating state information is not limited to the medical inspection device 10. That is, the transmission destination of the operating state information is arbitrary, and for example, the transmission function 453 may transmit the operating state information to the terminal apparatus or the like installed in the fixture room.

Subsequently, as illustrated in FIG. 16, the setting function 452 realized by the processing circuitry 45 of the management apparatus 40 regenerates the destination information (step S101). Specifically, the setting function 452 generates, as the destination information, the movement plan until moving to the destination of the medical inspection device 10 based on the inspection order OR1 acquired in step S39, the positional information of the medical inspection device 10, the floor map FM1 acquired in step S35, and the destination set in step S41. More specifically, for example, the setting function 452 generates the movement plan until moving to the inspection room which is the destination of the medical inspection device 10 via the fixture room for replacing the medical inspection fixture having the problem in the operating state.

Subsequently, as illustrated in FIG. 16, the transmission function 453 realized by the processing circuitry 45 of the management apparatus 40 retransmits the destination information (step S103). Specifically, the transmission function 453 retransmits the destination information regenerated in step S101 to the medical inspection device 10 via the communication interface 44.

The processing of step S105, step S107, step S109, and step S111 after step S101 is equivalent to the processing of step S57, step S65, step S67, and step S69 described above, and thus, description thereof is omitted. That is, the management apparatus 40 transmits the movement instruction to the fixture room (step S103), determines whether or not the medical inspection device 10 arrives at the fixture room (step S105), determines whether or not the collection of the medical inspection fixture is completed (step S109) in a case where the medical inspection device 10 arrives at the fixture room (step S107: Yes), and transmits the movement instruction to the inspection room (step S111) in a case where the collection of the medical inspection fixture is completed (step S109: Yes). Then, after step S111, the processing returns to step S77, and in step S83, the processing from step S77 is repeated until there is no problem in the operating state of the medical inspection device 10.

On the other hand, in a case where there is no problem in the operating state in step S83 (step S83: No), the setting function 452 realized by the processing circuitry 45 of the management apparatus 40 determines whether or not the medical inspection device 10 arrives at the inspection room (step S113). Specifically, the setting function 452 determines whether or not the medical inspection device 10 arrives at the inspection room by determining whether or not the positional information of the medical inspection device 10 is the position of the inspection room on the floor map FM1 based on the positional information acquired from the medical inspection device 10 and the floor map. Then, in step S113, in a case where the medical inspection device 10 does not arrive at the inspection room (step S113: No), the setting function 452 repeats step S113 and stands by.

On the other hand, in step S113, in a case where the medical inspection device 10 arrives at the inspection room (step S113: Yes), the notification function 456 realized by the processing circuitry 45 of the management apparatus 40 notifies that the medical inspection device 10 arrives (step S115). Specifically, the notification function 456 causes the medical inspection device 10 to notify that the medical inspection device 10 arrives by notifying that the medical inspection device 10 arrives via the communication interface 44. Note that, in step S111, although the notification function 456 causes the medical inspection device 10 to make a notification, the present embodiment is not limited thereto. For example, the notification function 456 may notify that the medical inspection device 10 arrives by causing the terminal apparatus possessed by the medical worker in the inspection room to make a notification.

Subsequently, as illustrated in FIG. 16, after step S115 or in a case where the medical inspection device 10 arrives at the destination in step S95 (step S95: Yes), the setting function 452 ends the acquisition of the positional information (step S117). In step S117, the acquisition of the positional information is ended, and thus, the movement instruction processing according to the present embodiment is ended.

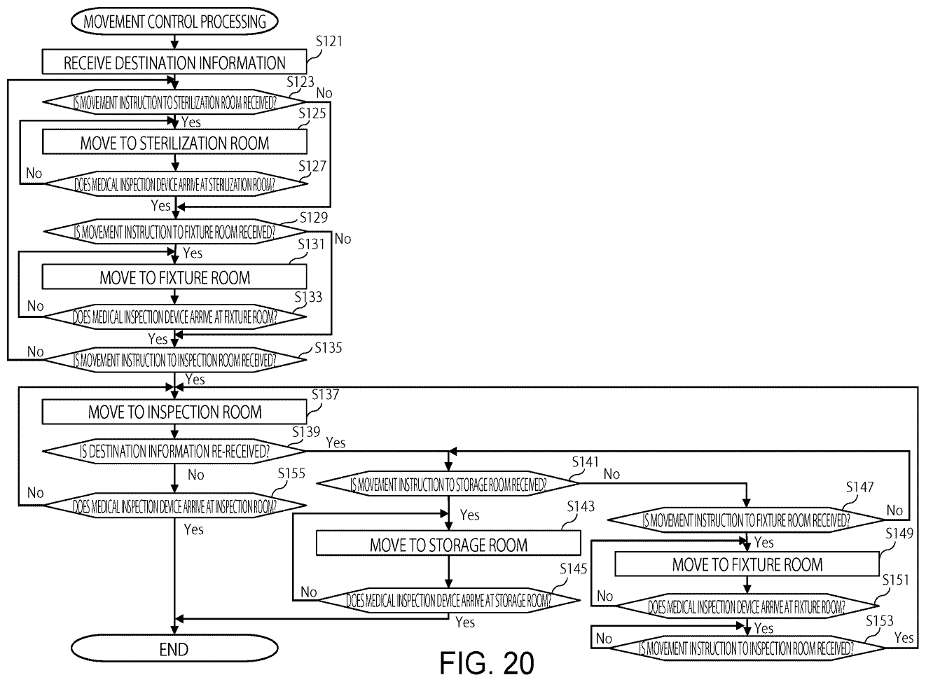

FIG. 20 is a flowchart for describing contents of the movement control processing executed by the medical inspection device 10 according to the first embodiment. In this movement control processing, in a case where the movement instruction is received from the management apparatus 40, the medical inspection device moves based on the destination information. For example, this movement control processing is processing executed in a case where the medical inspection device 10 receives the destination information from the management apparatus 40.

As illustrated in FIG. 20, first, the control function 145 realized by the processing circuitry 140 of the medical inspection device 10 receives the destination information (step S121). Specifically, the control function 145 receives, via the communication interface 135, the destination information transmitted by the management apparatus 40 in step S45 in FIG. 15 described above. In a case where the destination information is received, the control function 145 controls the support member 19 to move the display 15 and the input interface 17 to the unused position below the use position. As a result, since the medical inspection device 10 can move in a state where the size of the medical inspection device 10 is reduced, a possibility of coming in contact with a person or an object during movement can be reduced.

Subsequently, as illustrated in FIG. 20, the control function 145 realized by the processing circuitry 140 of the medical inspection device 10 determines whether or not the movement instruction to the sterilization room is received (step S123). Specifically, the control function 145 determines whether or not the movement instruction to the sterilization room transmitted by the management apparatus 40 is received in step S49 of FIG. 15 described above via the communication interface 135.

Then, in a case where the movement instruction to the sterilization room is received in step S123 (step S123: Yes), the control function 145 realized by the processing circuitry 140 of the medical inspection device 10 moves the medical inspection device 10 to the sterilization room (step S125). Specifically, the control function 145 controls the drive unit 139 based on the destination information received in step S121 to move the medical inspection device 10 to the sterilization room.

Subsequently, as illustrated in FIG. 20, the control function 145 realized by the processing circuitry 140 of the medical inspection device 10 determines whether or not the medical inspection device 10 arrives at the sterilization room (step S127). Specifically, the control function 145 determines whether or not the medical inspection device 10 arrives at the sterilization room based on the destination information received in step S121. Then, in step S127, in a case where the medical inspection device 10 does not arrive at the sterilization room (step S127: No), the control function 145 repeats the processing of steps S125 and S127 until the medical inspection device arrives at the sterilization room.

On the other hand, in a case where the medical inspection device 10 arrives at the sterilization room in step S127 (step S127: Yes), or in a case where the movement instruction to the sterilization room is not received in step S123 (step S123: No), the control function 145 realized by the processing circuitry 140 of the medical inspection device 10 determines whether or not the movement instruction to the fixture room is received (step S129). Specifically, the control function 145 determines whether or not the movement instruction to the fixture room transmitted by the management apparatus 40 is received in step S57 of FIG. 15 described above.

Then, in step S129, in a case where the movement instruction to the fixture room is received (step S129: Yes), the control function 145 realized by the processing circuitry 140 of the medical inspection device 10 moves the medical inspection device 10 to the fixture room (step S131). Specifically, the control function 145 controls the drive unit 139 based on the destination information received in step S121 to move the medical inspection device 10 to the fixture room.

Subsequently, as illustrated in FIG. 20, the control function 145 realized by the processing circuitry 140 of the medical inspection device 10 determines whether or not the medical inspection device 10 arrives at the fixture room (step S133). Specifically, the control function 145 determines whether or not the medical inspection device 10 arrives at the fixture room based on the destination information received in step S121. Then, in step S133, in a case where the medical inspection device 10 does not arrive at the fixture room (step S133: No), the control function 145 repeats the processing of steps S131 and S133 until the medical inspection device 10 arrives at the fixture room.

On the other hand, in a case where the medical inspection device 10 arrives at the fixture room in step S133 (step S133: Yes) or in a case where the movement instruction to the fixture room is not received in step S129 (step S129: No), the control function 145 realized by the processing circuitry 140 of the medical inspection device 10 determines whether or not the movement instruction to the inspection room is received (step S135). Specifically, the control function 145 determines whether or not the movement instruction to the inspection room transmitted by the management apparatus 40 is received in step S69 of FIG. 16 described above. Then, in a case where the movement instruction to the inspection room is not received in step S135 (step S135: No), the processing returns to step S123, and the processing from step S123 is repeated and stands by.

On the other hand, in a case where the movement instruction to the inspection room is received in step S135 (step S135: Yes), the control function 145 realized by the processing circuitry 140 of the medical inspection device 10 moves the medical inspection device 10 to the inspection room (step S137). Specifically, the control function 145 controls the drive unit 139 based on the destination information received in step S121 to move the medical inspection device 10 to the inspection room.

Subsequently, as illustrated in FIG. 20, the control function 145 realized by the processing circuitry 140 of the medical inspection device 10 determines whether or not the destination information is re-received (step S139). Specifically, the control function 145 determines whether or not the destination information transmitted by management apparatus 40 is received in step S93 or step S105 in FIG. 16 described above via the communication interface 135.

Then, in step S139, in a case where the destination information is re-received (step S139: Yes), the control function 145 realized by the processing circuitry 140 of the medical inspection device 10 determines whether or not the movement instruction to the storage room is received (step S141). Specifically, the control function 145 determines whether or not the movement instruction to the storage room transmitted by the management apparatus 40 in step S93 of FIG. 16 described above is received via the communication interface 135.

Then, in step S141, in a case where the movement instruction to the storage room is received (step S141: Yes), the control function 145 realized by the processing circuitry 140 of the medical inspection device 10 moves the medical inspection device 10 to the storage room (step S143). Specifically, the control function 145 controls the drive unit 139 based on the destination information re-received in step S139 to move the medical inspection device 10 to the storage room.

Subsequently, as illustrated in FIG. 20, the control function 145 realized by the processing circuitry 140 of the medical inspection device 10 determines whether or not the medical inspection device 10 arrives at the storage room (step S145). Specifically, the control function 145 determines whether or not the medical inspection device 10 moves to the storage room based on the destination information re-received in step S139. Then, in step S145, in a case where the medical inspection device 10 does not arrive at the storage room (step S145: No), the control function 145 repeats the processing of steps S143 and S145 until the medical inspection device 10 arrives at the storage room.

On the other hand, in a case where the movement instruction to the storage room is not received in step S141, it is determined whether or not the movement instruction to the fixture room is received (step S147). Specifically, the control function 145 determines whether or not the movement instruction to the fixture room transmitted by the management apparatus 40 in step S105 of FIG. 16 is received. Then, in a case where the movement instruction to the fixture room is not received in step S147 (step S147: No), the control function 145 repeats the processing of steps S141 and S147 and stands by until the movement instruction to the storage room or the movement instruction to the fixture room is received.

On the other hand, in a case where the movement instruction to the fixture room is received in step S147 (step S147: Yes), the control function 145 realized by the processing circuitry 140 of the medical inspection device 10 moves to the fixture room (step S149). Specifically, the control function 145 controls the drive unit 139 based on the destination information re-received in step S143 to move the medical inspection device 10 to the fixture room.

Subsequently, as illustrated in FIG. 20, the control function 145 realized by the processing circuitry 140 of the medical inspection device 10 determines whether or not the medical inspection device 10 arrives at the fixture room (step S151). Specifically, the control function 145 determines whether or not the medical inspection device 10 arrives at the fixture room based on the destination information re-received in step S143. Then, in step S151, in a case where the medical inspection device 10 does not arrive at the fixture room (step S151: No), the control function 145 repeats the processing of steps S149 and S151 until the medical inspection device 10 arrives at the fixture room.

On the other hand, in a case where the medical inspection device 10 arrives at the fixture room in step S151 (step S151: Yes), the control function 145 realized by the processing circuitry 140 of the medical inspection device 10 determines whether or not the movement instruction to the inspection room is received (step S153). Specifically, the control function 145 determines whether or not the movement instruction to the inspection room is received by determining whether or not the movement instruction to the inspection room transmitted by the management apparatus 40 in step S111 of FIG. 16 described above is received. Then, in a case where the movement instruction to the inspection room is not received in step S153 (step S153: No), the control function 145 realized by the processing circuitry 140 of the medical inspection device 10 repeats the processing of step S153 and stands by until the movement instruction to the inspection room is received. On the other hand, in a case where the movement instruction to the inspection room is received in step S153 (step S153: Yes), the processing returns to step S137, and the processing from step S137 is repeated.

On the other hand, in a case where the destination information is not re-received in step S139 (step S139: No), the control function 145 realized by the processing circuitry 140 of the medical inspection device 10 determines whether or not the medical inspection device 10 arrives at the inspection room (step S155). Specifically, the control function 145 determines whether or not the medical inspection device 10 arrives at the inspection room based on the destination information received in step S121. Then, in a case where the medical inspection device does not arrive at the inspection room in step S155 (step S155: No), the control function 145 repeats the processing of steps S137, S139, and S155 until the medical inspection device arrives at the inspection room.

On the other hand, in a case where the medical inspection device 10 arrives at the inspection room in step S155 (step S155: Yes), or in a case where the medical inspection device 10 arrives at the storage room in step S145 (step S145: Yes), the movement control processing according to the present embodiment is ended.

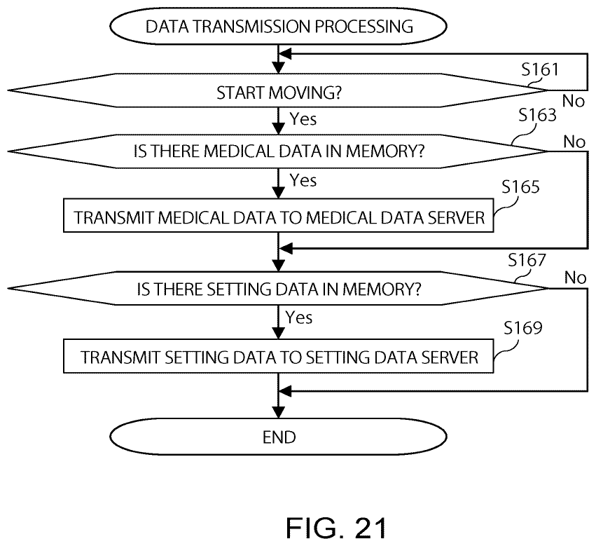

FIG. 21 is a flowchart for describing contents of data transmission processing executed by the medical inspection device 10 according to the first embodiment. In this data transmission processing, in a case where the medical inspection device 10 starts moving, the medical data is transmitted to the medical data server 20, and the setting data is transmitted to the setting data server 30. For example, this data transmission processing is processing executed in a case where the medical inspection device 10 starts moving.

As illustrated in FIG. 21, first, the data transmission function 142 realized by the processing circuitry 140 of the medical inspection device 10 determines whether or not the medical inspection device 10 starts moving (step S161). Then, in step S161, in a case where the medical inspection device 10 does not start moving (step S161: No), the data transmission function 142 repeats the processing of step S161 and stands by until the medical inspection device 10 starts moving.

On the other hand, in step S161, in a case where the medical inspection device 10 starts moving (step S161: Yes), the data transmission function 142 realized by the processing circuitry 140 of the medical inspection device 10 determines whether or not there is the medical data in the memory 134 (step S163). Specifically, the data transmission function 142 determines whether or not there is the medical data in the memory 134 by determining whether or not the medical data collected in the inspection before movement is stored in the memory 134.

Then, in a case where there is the medical data in the memory 134 (step S163: Yes), the data transmission function 142 realized by the processing circuitry 140 of the medical inspection device 10 transmits the medical data to the medical data server 20 (step S165). Specifically, the data transmission function 142 transmits the medical data stored in the memory 134 to the medical data server 20 via the communication interface 135. That is, the medical inspection device 10 transmits the medical data collected in the inspection to the medical data server 20 while moving to the destination after the start of the movement.

On the other hand, in step S163, when there is no medical data in the memory 134 (step S163: No), or after step S165, the data transmission function 142 realized by the processing circuitry 140 of the medical inspection device 10 determines whether or not there is the setting data in the memory 134 (step S167). Specifically, the data transmission function 142 determines whether or not there is the setting data in the memory 134 by determining whether or not the setting data used in the inspection before movement is stored in the memory 134.

Then, in step S167, in a case where there is the setting data in the memory 134 (step S167: Yes), the data transmission function 142 realized by the processing circuitry 140 of the medical inspection device 10 transmits the setting data to the setting data server 30 (step S169). Specifically, the data transmission function 142 transmits the setting data stored in the memory 134 and used in the previous inspection to the setting data server 30 via the communication interface 135.

After step S169 or in a case where there is no setting data in the memory 134 in step S167 (step S167: No), the data transmission processing according to the present embodiment is ended.

FIG. 22 is a flowchart for describing contents of diagnosis processing executed by the medical inspection device 10 according to the first embodiment. In this diagnosis processing, in a case where the diagnosis instruction of the operating state transmitted from the management apparatus 40 is received, the operating state is diagnosed, and the diagnosis result of the operating state is transmitted. For example, this diagnosis processing is processing executed in a case where the diagnosis instruction of the operating state is received.

As illustrated in FIG. 22, first, the diagnosis function 143 realized by the processing circuitry 140 of the medical inspection device 10 determines whether or not the diagnosis instruction of the operating state is received (step S171). Specifically, the diagnosis function 143 determines whether or not the diagnosis instruction of the operating state transmitted in step S77 of FIG. 16 described above is received. Then, in step S171, in a case where the diagnosis instruction of the operating state is not received (step S171: No), the diagnosis function 143 repeats the processing of step S171 and stands by until the diagnosis instruction of the operating state is received.

On the other hand, in a case where the diagnosis instruction of the operating state is received in step S171 (step S171: Yes), the diagnosis function 143 realized by the processing circuitry 140 of the medical inspection device 10 diagnoses the operating state (step S173). Specifically, diagnosis function 143 diagnoses the operating state of the apparatus body 13 and/or the ultrasonic probe 11 electrically connected to the apparatus body 13. In the present embodiment, since the management apparatus 40 transmits the diagnosis instruction of the operating state to the medical inspection device 10 while moving to the destination, the medical inspection device 10 diagnoses the operating state of the apparatus body 13 and/or the ultrasonic probe 11 electrically connected to the apparatus body 13 while moving to the destination.

Subsequently, as illustrated in FIG. 22, the diagnosis function 143 realized by the processing circuitry 140 of the medical inspection device 10 transmits the diagnosis result of the operating state (step S175). Specifically, the diagnosis function 143 transmits the diagnosis result of the operating state to the management apparatus 40 via the communication interface 44.

In step S175, the diagnosis processing according to the present embodiment is ended by transmitting the diagnosis result of the operating state.

As described above, according to the medical inspection device management system 1 according to the present embodiment, the management apparatus 40 selects the available medical inspection device 10 based on the inspection order OR1, sets the destination of the selected medical inspection device 10, and transmits the destination information regarding the destination to the selected medical inspection device 10, and the medical inspection device 10 having the self-traveling function of moving to the destination moves to the destination based on the destination information. Therefore, it is possible to dispose the medical inspection device at the destination without requiring the labor of the medical worker such as a staff member. That is, according to the medical inspection device management system 1 of the present embodiment, it is possible to improve the productivity of the medical worker while effectively utilizing the assets and resources in the hospital.

### Modification 1

In the medical inspection device management system 1 according to the first embodiment, the user directly inputs the inspection date and time as the inspection date and time included in the inspection order OR1 via the input interface of the terminal apparatus 50. However, the inspection date and time included in the inspection order OR1 can be input as the inspection date and time of the inspection order OR1 by selecting a time zone desired to be used by the user from a reservation status list showing reservation statuses of the medical inspection devices 10 displayed on a display of the terminal apparatus 50 via the input interface of the terminal apparatus 50.

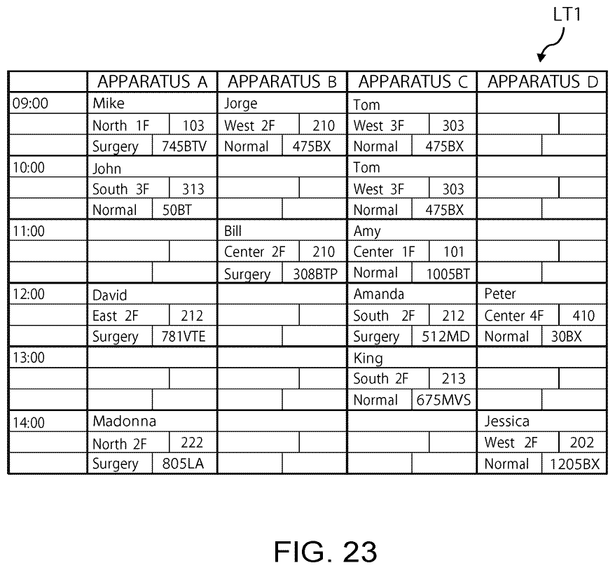

FIG. 23 is a diagram illustrating an example of a reservation status list displayed on a display of a terminal apparatus 50 of a medical inspection device management system 1 according to Modification 1. As illustrated in FIG. 23, a management apparatus 40 displays a reservation status list LT1 on the display of the terminal apparatus 50. In the example illustrated in FIG. 23, the reservation status list LT1 shows a reservation status in each time zone for each medical inspection device 10. The user inputs an inspection date and time of the inspection order OR1 by checking an available time zone for each medical inspection device 10 from the reservation status list LT1 and selecting a time zone desired to be used from the reservation status list LT1 displayed on the display of the terminal apparatus 50 via the input interface of the terminal apparatus 50.

As described above, in the medical inspection device management system 1 according to the present modification, the inspection date and time of the inspection order OR1 is input by displaying the reservation status list LT1 on the display of the terminal apparatus 50 and accepting the selection of the time zone desired to be used by the user with respect to the displayed reservation status list LT1. Therefore, it is possible to select an available time zone of the medical inspection device 10 as the inspection date and time. That is, in the reservation of the medical inspection device 10, a possibility of changing the inspection date and time can be reduced, and the convenience of the user can be improved.

### Modification 2

In the medical inspection device management system 1 according to the first embodiment described above, the desired medical inspection device information regarding the medical inspection device 10 desired to be used by the user is included in the inspection order OR1, and the management apparatus 40 can select the medical inspection device 10 based on the inspection order including the desired medical inspection device information.

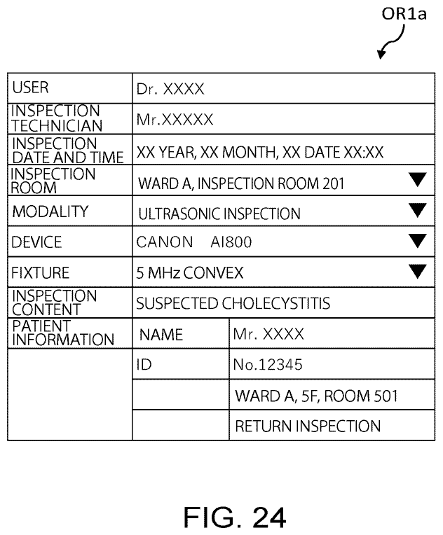

FIG. 24 is a diagram illustrating another example of an inspection order received by a management apparatus 40 according to Modification 2, and is a diagram corresponding to FIG. 14 in the first embodiment described above. In the example illustrated in FIG. 24, an inspection order OR1a includes a name of the device as the desired medical inspection device information in addition to the user, the inspection date and time, the inspection room, the modality, the inspection content, the patient information including the patient name and the patient ID, the information for identifying whether the patient is the outpatient or the inpatient, and the inspection number information.

Thus, for example, in step S13 in the reservation processing of FIG. 13, the selection function 451 in the processing circuitry 45 of the management apparatus 40 determines whether or not the inspection order OR1a is acceptable by determining whether or not the device of the desired medical inspection device information can be used at the inspection date and time included in the inspection order. Then, in a case where the inspection order OR1a is not acceptable in step S13, the selection function 451 notifies the user of a change notification of the inspection date and time by notifying the user that the reservation cannot be made in step S15 and notifying the user of the information regarding the time zone in which the inspection device can be used. On the other hand, in a case where the inspection order OR1a is acceptable in step S13, the selection function 451 may select the device of the desired medical inspection device information as the available medical inspection device in step S19. With this configuration, since the user can use the same medical inspection device 10 for each inspection, the convenience of the user can be improved.

### Modification 3

In the medical inspection device management system 1 according to the first embodiment, the management apparatus 40 can select the medical inspection device 10 based on the pieces of inspection number information included in the inspection orders OR1 and OR1a. For example, as illustrated in FIG. 10, in a case where the inspection orders OR1 and OR1a include the return inspection as the inspection number information, in step S13 in the reservation processing of FIG. 13, it is determined whether or not the inspection orders OR1 and OR1a are acceptable by referring to the information regarding the previous inspection of the patient included in the patient information and determining whether or not the medical inspection device 10 used in the previous inspection can be used at the inspection date and times included in the inspection orders OR1 and OR1a. Then, in a case where the inspection orders OR1 and OR1a are acceptable, the selection function 451 may select the medical inspection device 10 used in the previous inspection as the available medical inspection device in step S19 in the reservation processing of FIG. 13. With this configuration, it is possible to reduce a difference in image output due to a difference in the medical inspection device 10 used for the inspection.

Note that, in the medical inspection device management system 1 according to Modification 3, in a case where the inspection orders OR1 and OR1a are not acceptable in step S13 in the reservation processing of FIG. 13, the selection function 451 may notify that the reservation cannot be made in step S17 in the reservation processing of FIG. 13, and may notify an available time zone of the medical inspection device 10 used in the previous inspection, or may notify that the medical inspection device 10 different from the medical inspection device 10 used in the previous inspection is selectable at the inspection date and times included in the inspection orders OR1 and OR1a. By performing these notifications, the user can change the inspection orders, and the management apparatus 40 can re-receive the inspection orders OR1 and OR1a in which the information regarding the inspection date and time or the medical inspection device 10 is changed in step S17 in the reservation processing of FIGS. 9A and 9B.

### Modification 4

In the medical inspection device management system 1 according to the first embodiment described above, in a case where the plurality of inspections orders OR1 and OR1a including the same inspection date and time are received, the management apparatus 40 can also reserve the inspection by giving priority to the outpatient. In this case, the inspection orders OR1 and OR1a received in step S11 of FIG. 13 include information for identifying whether the patient is the outpatient or the inpatient. For example, in a case where the plurality of inspections orders OR1 and OR1a including the same inspection date and time are received, the management apparatus 40 can reserve the inspection by giving priority to the inspection orders OR1 and OR1a of the outpatient.

### Modification 5

In the medical inspection device management system 1 according to the first embodiment described above, in a case where the management apparatus 40 receives the inspection orders OR1 and OR1a including information indicating that the patient is the inpatient, it is also possible to determine whether or not the small medical inspection device 10 among the available medical inspection devices 10 can be used. For example, in a case where the management apparatus 40 includes information that the inspection orders OR1 and OR1a received in step S11 of FIG. 13 are the inpatient, the management apparatus 40 determines whether or not the inspection orders OR1 and OR1a are acceptable by determining whether or not the small medical inspection device 10 among the available medical inspection devices 10 can be used at the inspection date and times included in the inspection orders OR1 and OR1a in step S13 of FIG. 13. Then, in a case where the inspection orders OR1 and OR1a are acceptable in step S13 of FIG. 13, the selection function 451 selects the small medical inspection device 10 as the available medical inspection device in step S19 of FIG. 13. With such a configuration, even in a case where the inspection is performed on a bedside of the inpatient, the medical inspection device 10 can be introduced into the bedside of the inpatient.

### Modification 6

In the medical inspection device management system 1 according to the first embodiment described above, the management apparatus 40 can also select the available medical inspection device 10 based on the information regarding the inspection room. The information regarding the inspection room is, for example, information regarding a size of the inspection room. For example, in step S19 in the reservation processing of FIG. 13, the selection function 451 may select the available medical inspection device corresponding to the size of the inspection room based on the information regarding the inspection room together with the inspection date and time.

### Modification 7

In the medical inspection device management system according to the first embodiment described above, in a case where the inspection order includes used fixture information regarding a fixture used in the inspection, the management apparatus 40 can select the available medical inspection device 10 based on the used fixture information. The used fixture information is information regarding the fixture used in the inspection, and includes, for example, a name of the ultrasonic probe. Then, in a case where the used fixture information is included in the inspection order OR1a received in step S11 of FIG. 13, in step S15 in the reservation processing, the selection function 451 may select, as the available medical inspection device 10, the medical inspection device 10 that can use the fixture based on the used fixture information together with the inspection date and time. More specifically, for example, in a case where an acoustic coupler used in a mammary gland inspection, a rheumatism inspection, or an elastography inspection is included as the used fixture information in the inspection order, the selection function 451 selects the medical inspection device 10 that can use the acoustic coupler. In addition, as illustrated in FIG. 24, for example, in a case where “5 MHz convex” is included as the used fixture information in the inspection order OR1a, the selection function 451 selects the available medical inspection device 10 of “5 MHz convex”.

### Modification 8

In the medical inspection device management system 1 according to the first embodiment described above, the management apparatus 40 can make a reservation of the medical inspection device 10 and a reservation of the inspection technician. In this case, the inspection order OR1a received in step S11 of FIG. 13 also includes the desired inspection technician information. Specifically, as illustrated in FIG. 24, the inspection order OR1a includes the name of the inspection technician as the desired inspection technician information. In this case, in a case where the selection function 451 receives the inspection order OR1a in the reservation processing, the management apparatus 40 determines whether or not the inspection order OR1a is acceptable by determining whether or not the medical inspection device 10 can be used at the inspection date and time in step S13 and determining whether or not the inspection can be performed by the inspection technician included in the desired inspection technician information. Then, in a case where the inspection order OR1a is acceptable, the selection function 451 may provisionally reserve the medical inspection device 10 and the inspection technician in step S21 by selecting the available medical inspection device 10 and selecting the inspection technician in step S19. With such a configuration, it is possible to suppress variation in the inspection result due to a difference in the inspection technician to perform the inspection.

### Other Modifications of First Embodiment

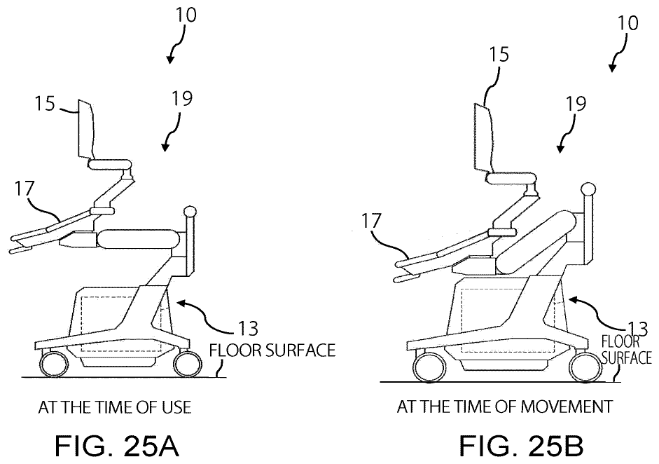

In the first embodiment described above, the medical inspection device 10 may move without folding the display 15 during movement. FIGS. 25A and 25B are schematic views illustrating another example of the appearance at the time of use (FIG. 25A) and at the time of movement (FIG. 25B) of the medical inspection device 10 according to the first embodiment, and are diagrams corresponding to FIGS. 9A and 9B in the above-described first embodiment. As illustrated in FIG. 25B, when the medical inspection device 10 moves, the medical inspection device 10 may move without folding the display 15.

Note that the word “processor” used in above descriptions means circuits such as, for example, a Central Processing Unit (CPU), a Graphics Processing Unit (GPU), an Application Specific Integrated Circuit (ASIC), a programmable logic device (for example, a Simple Programmable Logic Apparatus (SPLD), a Complex Programmable Logic Apparatus (CPLD), and a Field Programmable Gate Array (FPGA)). The processor executes functions by reading and executing programs stored in the memory 8. Note that programs may be configured to be directly integrated in the processor instead of being storing in the memory 8. In this case, the processor realizes functions by reading and executing programs stored in the circuitry. Note that the processor is not limited to the case arranged as a single processor circuit, but may be configured as a single processor by combining a plurality of independent circuits to realize functions. Furthermore, a plurality of component elements in FIG. 1 may be integrated into one processor to realize the functions.

While certain embodiments have been described, these embodiments have been presented by way of example only and are not intended to limit the scope of the inventions. The embodiments may be in a variety of other forms. Furthermore, various omissions, substitutions and changes may be made without departing from the spirit of the inventions. The embodiments and their modifications are included in the scope and the subject matter of the invention, and at the same time included in the scope of the claimed inventions and their equivalents.

1. A medical inspection device management system, comprising:
one or a plurality of medical inspection devices having a self-traveling function of moving to a destination; and
a management apparatus,
wherein the management apparatus includes a first processing circuitry configured to
select an available medical inspection device based on an inspection order,
set the destination of the medical inspection device, and
transmit destination information regarding the destination to the medical inspection device.

2. The medical inspection device management system according to claim 1, wherein
the inspection order includes at least medical inspection device information regarding the medical inspection device and an inspection date and time, and
the first processing circuitry is further configured to select the available medical inspection device based on the medical inspection device information and the inspection date and time.

3. The medical inspection device management system according to claim 1, wherein the inspection order includes at least one of desired inspection technician information regarding an inspection technician desired by a doctor to perform an inspection, desired medical inspection device information regarding the medical inspection device desired by a user to use, used fixture information regarding a medical inspection fixture used in the inspection by the user, inspection number information regarding the number of times of the inspection, and patient information regarding a patient on which the inspection is performed.

4. The medical inspection device management system according to claim 3, wherein the first processing circuitry is further configured to select the medical inspection fixture necessary for the inspection from among a plurality of the medical inspection fixtures based on the inspection order.

5. The medical inspection device management system according to claim 4, wherein the first processing circuitry is further configured to transmit medical inspection fixture information regarding the selected medical inspection fixture to the medical inspection device.

6. The medical inspection device management system according to claim 1, wherein the first processing circuitry is further configured to select setting data necessary for the inspection from among a plurality of pieces of setting data which are pieces of data for each user based on the inspection order.

7. The medical inspection device management system according to claim 6, wherein the first processing circuitry is further configured to transmit setting data information regarding the selected setting data to the medical inspection device.

8. The medical inspection device management system according to claim 1, wherein the first processing circuitry is further configured to select the medical inspection device based on operating status information regarding an operating status of the medical inspection device in a case where there is a plurality of the available medical inspection devices.

9. The medical inspection device management system according to claim 7, wherein the medical inspection device includes a second processing circuitry configured to acquire setting data based on the setting data information from a setting data server while moving to the destination.

10. The medical inspection device management system according to claim 1, wherein the medical inspection device includes a second processing circuitry configured to transmit medical data collected in the inspection to a medical data server while moving to the destination.

11. The medical inspection device management system according to claim 4, wherein the medical inspection device includes a second processing circuitry configured to diagnose an operating state of the medical inspection device body and/or the medical inspection fixture electrically connected to the medical inspection device body while moving to the destination.

12. The medical inspection device management system according to claim 11, wherein the first processing circuitry is further configured to
receive a diagnosis result of the operating state diagnosed by the medical inspection device, and
determine the operating state based on the diagnosis result.

13. The medical inspection device management system according to claim 1, wherein the first processing circuitry is further configured to notify that the medical inspection device arrives at the destination in a case where the medical inspection device arrives at the destination.

14. The medical inspection device management system according to claim 1, wherein the medical inspection device includes:
a battery; and
a non-contact power receiver configured to receive power in a non-contact manner from a non-contact power supply that supplies power in a non-contact manner so as to supply power to the battery while moving to the destination.

15. An ultrasonic diagnosis apparatus, comprising:
an apparatus body having a self-traveling function of moving to a destination, and configured to generate an ultrasonic image;
an input interface configured to receive an input operation of operating the apparatus body;
a display configured to display the ultrasonic image;
a support member configured to movably support the input interface and the display with respect to the apparatus body; and
a processing circuitry configured to control the support member such that the input interface and the display are moved to a use position based on user information regarding a user who uses the ultrasonic diagnosis apparatus at the destination and the input interface and the display are moved to an unused position below the use position at the time of movement to the destination.

16. A management apparatus, comprising:
a processing circuitry configured to
select an available medical inspection device among one or a plurality of medical inspection devices having a self-traveling function of moving to a destination based on an inspection order,
set the destination of the medical inspection device, and
transmit destination information regarding the destination to the medical inspection device.
